(19) United States
(12) Patent Application Publication
(10) Pub. No.: US 2025/0268833 A1
(43) Pub. Date: Aug. 28, 2025

(54) Zwitterionic Hydrogels and Methods of Making and Using Same for Protein Therapy

(71) Applicant: Board of Regents for the Oklahoma Agricultural and Mechanical Colleges, Stillwater, OK (US)

(72) Inventors: Amir ERFANI, Stillwater, OK (US); Nicholas H. FLYNN, Edmond, OK (US); Climt P. AICHELE, Stillwater, OK (US); Joshua D. RAMSEY, Stillwater, OK (US)

(21) Appl. No.: 18/043,452

(22) PCT Filed: Aug. 27, 2021
(86) PCT No.: PCT/US2021/048020

§ 371 Date: Feb. 28, 2023

Related U.S. Application Data
(60) Provisional application No. 63/071,932, filed on Aug. 28, 2020.

(51) Int. Cl. A61K 9/48

(52) U.S. Cl. CPC A61K 9/4816

(57) ABSTRACT

Biocompatible zwitterionic hydrogels, methods for making the hydrogels and polymers making up the hydrogels, and methods of use of the hydrogels are disclosed. The zwitterionic hydrogels are loaded with at least one active biomacromolecule that can be encapsulated or immobilized within the hydrogel.

### INCORPORATION BY REFERENCE

This application claims the benefit of provisional application U.S. Ser. No. 63/071,932, filed Aug. 28, 2020. The entire contents of the before-referenced application are expressly incorporated herein by reference.

### BACKGROUND

Recently, hydrogels have gained attention for use as delivery vehicles in biomedical applications, agriculture, and the food industry. Hydrogels can potentially be used for the delivery of both small molecule (i.e., localized chemotherapy) and macromolecule (i.e., antibodies, hormones) pharmaceuticals. With respect to macromolecules, challenging issues exist, for example, the solubility of the protein and the stability of the macromolecule at body temperature, as well as its efficacy and immunogenicity.

Zwitterionic polymers are composed of monomers that have an equal number of cationic and anionic charges that produce a neutrally charged, superhydrophilic polymer. Zwitterionic polymers have been shown to prevent biofouling and protein adsorption. This biomimic strategy is inspired by protein stabilization using zwitterionic moieties in several living organisms. In organisms such as sugar beets or saline soil herbs, zwitterionic inner salts such as sulfobetaine or carboxybetaine act as natural osmolytes and increase the protein conformational stability. Zwitterionic polymers have natural osmolytes as their repeating units. Zwitterionic hydrogels (crosslinked polymers with hydrating water) can have superhydrophilic characteristics and increase protein stability while maintaining high water content, which is important for protein stability and activity.

The superhydrophilic properties of zwitterions have unique effects on protein folding and stabilization. Protein folding is driven primarily by hydrophobic interactions (as well as, to a lesser degree, electrostatic interactions, and hydrogen bonding) and can be guided by various molecular chaperones that navigate the protein structure past local energy minima to the final folded form. Because of their osmolyte nature, zwitterionic molecules have been studied as chemical chaperones. For example, the use of non-detergent sulfobetaines (SB) inner-salts as cosolvents has been investigated and shown to enhance refolding of denatured galactosidase up to 80-fold and denatured lysozyme up to 12-fold (probed by the protein activity). See Goldberg, M. E.; Expert-Bezangon, N.; Vuillard, L.; Rabilloud, T., Non-detergent sulphobetaines: A new class of molecules that facilitate in vitro protein renaturation. Folding and Design 1996, 1 (1), 21-27. In a similar study, the refolding of four different proteins by non-detergent SB was evaluated and new SB moieties were designed to optimize the chaperone characteristics of SB, reaching high renaturation yields. See Expert-Bezangon, N.; Rabilloud, T.; Vuillard, L.; Goldberg, M. E., Physical-chemical features of non-detergent sulfobetaines active as protein-folding helpers. Biophysical Chemistry 2003, 100 (1-3), 469-479.

Maintaining protein (e.g., enzyme, monoclonal antibody) conformation and function is essential for them to be successfully utilized as therapeutics, biocatalysts, or biosensors. As such, there is a need for materials that increase protein stability and prevent undesired non-specific protein binding, aggregation, and structural change.

Enzymes are useful in industrial chemistry, biosensors, and medicine, specifically for therapeutic uses. Enzyme immobilization for therapeutic purposes has been studied for cancer therapy, enzyme replacement therapy, and treatment of cystic fibrosis, organophosphate intoxication, and gastrointestinal diseases. However, the adoption and use of enzyme or protein therapy in therapeutic settings has been hindered by the poor stability and short half-life of proteins in vivo. Further, enzymes are expensive, incompatible with many environments, and suffer from poor reusability. Protein stability can be limited at the elevated temperatures required in processes or biological environments.

In the immobilization of proteins, such as on hydrogels, undesired interactions between the proteins and the polymeric networks can result in unfolding and deactivation of the protein. A major challenge is that many materials, such as silicon and other hydrophobic coatings, are inherently incompatible with proteins and lead to protein binding and aggregation, structural changes, and denaturing. Additionally, protein structure and function have a significant impact on its immunogenicity and can result in unwanted immunogenic reactions. Similarly, biofouling can hinder the specific bio-affinity for biosensors.

The way in which proteins are immobilized also play a major role in the loss of protein activity due to random orientation and structural deformations that can occur. The current strategies to overcome such protein immobilization are physical, covalent, and bio-affinity binding. Covalent immobilization can add a spacer arm length between the protein and the immobilization matrix or zero-length crosslinking can be used.

Protease enzymes are commonly used industrial enzymes. Chymotrypsin (ChT) is a serine protease enzyme with histidine 57, aspartate 102, and serine 195 acting as its active sites. ChT has a limited stability and short half-life at conditions in which it is active. Furthermore, enzymes such as ChT have applications in organic chemistry. As many reactions are carried out in organic solutions, it is important to develop technologies that enable the use of active enzymes in organic media. More specifically, ChT has been used to synthesize peptides in organic media; yet like many other enzymes, ChT is not stable in organic media and has limited activity in hydrophobic environments. Additionally, protease enzymes such as ChT are gaining significant attention in food industry for applications such as reduction of protein allergy, tenderization, and easier digestible foods, especially where the enzymes must have and maintain activity in harsh conditions, such as at elevated temperatures.

Improved biocompatible hydrogels, methods for making, and use of the same in protein-based therapies may be used to immobilize enzymes for their use in therapeutics, biocatalysts, or biosensors, for example. It is to such improved biocompatible hydrogel compositions, methods, and uses that the presently disclosed and/or claim invention and technology is directed.

### BRIEF DESCRIPTION OF THE DRAWINGS

The accompanying drawings, which are incorporated in and constitute a part of this specification, illustrate one or more implementations described herein and, together with the description, explain these implementations. The drawings are not intended to be drawn to scale, and certain features and certain views of the figures may be shown exaggerated, to scale, or in schematic form in the interest of clarity and conciseness. Not every component may be labeled in every drawing. Like reference numerals in the figures may represent and refer to the same or similar element or function.

FIG. 1 is a schematic representation of the preparation of exemplary zwitterionic microgels (microscale hydrogels), specifically microscale poly(carboxybetaine), pCB, using inverse emulsion polymerization synthesized in accordance with the presently disclosed inventive concept(s).

FIG. 2 is an SEM micrograph mage of the lyophilized (freeze-dried) microscale hydrogels (no enzyme, or blank) at 1000× magnification, synthesized in accordance with the presently disclosed inventive concept(s).

FIGS. 3A-3B are SEM micrograph images of the lyophilized (freeze-dried) microscale hydrogels at 10000× magnification. FIG. 3A depicts exemplary hydrogels with no enzyme (blank). FIG. 3B depicts enzyme (chymotrypsin) immobilized hydrogel.

FIGS. 4A-4B are confocal microscopy images of exemplary fluorescently labeled enzyme (chymotrypsin) immobilized microscale hydrogels. Chymotrypsin was fluorescently tagged using FITC. FIG. 4A depicts magnification of 20×; FIG. 4B depicts magnification of 60×.

FIG. 5 is a graphical representation of the ultraviolet (UV) spectra results of the free chymotrypsin (Cht), ChT (enzyme) immobilized hydrogels, and blank pCB hydrogel.

FIGS. 6A-6B are graphical representations of the reusability of chymotrypsin immobilized within poly(carboxybetaine). FIG. 6A depicts the enzymatic activity for cycles of reusing the enzyme; FIG. 6B depicts the enzymatic activity for cycles of drying and rehydration of the enzyme immobilized hydrogel and the free enzyme.

FIGS. 7A-7B are graphical representations of the esterase enzymatic activity of chymotrypsin immobilized within pCB hydrogels at different conditions compared with free chymotrypsin in solution. FIG. 7A depicts the effect of pH, measured at constant temperature of 40° C. and normalized for pH=7.5; FIG. 7B depicts the effect of temperature, measured at constant pH of 7.5.

FIGS. 8A-8D are SEM images of exemplary freeze-dried hydrogels. 8A-8B: Hydrogel synthesized using bulk polymerization technique. 8C-8D: Hydrogel beads formed using inverse emulsion polymerization.

FIG. 9 is a graphical representation of the equilibrium water content (PBS as the water phase) of exemplary zwitterionic hydrogels with different crosslinking densities prepared using the bulk polymerization technique.

FIG. 10 is a graphical representation of the effects of temperature on equilibrium water content of zwitterionic pSB hydrogels.

FIG. 11 is a graphical representation of the ion responsivity of exemplary zwitterionic poly(sulfobetaine) (pSB) hydrogels.

FIGS. 12A-12B depicts ¹H NMR spectra of zwitterionic hydrogel. 12A: Low crosslink density (1:50 molar); 12B: High crosslink density (1:5 molar).

FIGS. 13A-13B are confocal microscopy images of exemplary fluorescently labeled protein (albumin) immobilized microscale hydrogels. FIG. 13B depicts the averaged projection of a series of optical sections at different depths of the hydrogel bead.

FIG. 14 is a graphical representation of the amount of protein loaded inside exemplary hydrogels, which was evaluated after release of FITC-albumin inside PBS.

FIGS. 15A-15B are graphical representations of release plots for three different zwitterionic pSB hydrogel crosslinking densities. 15A: Total amount of BSA released per mass of the solid hydrogel (excluding water). 15B: Normalized release of BSA for different crosslinking densities.

FIG. 16 is a graphical representation of the enzymatic activities of the released proteins of FIGS. 15A-15B.

FIGS. 17A-17B are graphical representations of BSA aggregation when exposed to 70° C. for 0-8 hours (17A) and BSA enzymatic activity after being exposed to 70° C. (17B). The “*” denotes statistically significant changes between the two data points.

FIGS. 18A-18B are schematic representations of exemplary zwitterionic pCB-TTEGDA and its hydrolytic degradation.

FIGS. 19A-C are SEM micrograph images that show the biodegradability of the microgels.

FIG. 20 Illustrates the cytotoxicity and immunogenicity experiments on exemplary biodegradable zwitterionic pCB microgels.

FIGS. 21A-B illustrate Antibody (Ab) release from exemplary zwitterionic microgels and the activity of released Ab evaluated using human IgG ELISA kit. FIG. 21A shows Ab release at 40° C. for three different crosslinking densities (fluorescently labelled IgG). FIG. 21B shows the activity of the released Abs for the Ab released during the early release stage (i.e., within the first 18 hours). FIG. 21C shows activity of the Abs released during the slow-release stage (i.e., 18 to 120 hours).

### DETAILED DESCRIPTION

Before explaining at least one embodiment of the present disclosure in detail, it is to be understood that the present disclosure is not limited in its application to the details of construction and the arrangement of the components or steps or methodologies set forth in the following description or illustrated in the drawings. The present disclosure is capable of other embodiments or of being practiced or carried out in various ways. Also, it is to be understood that the phraseology and terminology employed herein is for the purpose of description and should not be regarded as limiting.

Unless otherwise defined herein, technical terms used in connection with the present disclosure shall have the meanings that are commonly understood by those of ordinary skill in the art. Further, unless otherwise required by context, singular terms shall include pluralities and plural terms shall include the singular.

All patents, published patent applications, and non-patent publications mentioned in the specification are indicative of the level of skill of those skilled in the art to which the present disclosure pertains. All patents, published patent applications, and non-patent publications referenced in any portion of this application are herein expressly incorporated by reference in their entirety to the same extent as if each individual patent or publication was specifically and individually indicated to be incorporated by reference.

All of the articles and/or methods disclosed herein can be made and executed without undue experimentation in light of the present disclosure. While the articles and methods of the present disclosure have been described in terms of preferred embodiments, it will be apparent to those of ordinary skill in the art that variations may be applied to the articles and/or methods and in the steps or in the sequence of steps of the method(s) described herein without departing from the concept, spirit and scope of the present disclosure. All such similar substitutes and modifications apparent to those skilled in the art are deemed to be within the spirit, scope and concept of the present disclosure.

The following detailed description refers to the accompanying drawings. The same reference numbers in different drawings may identify the same or similar elements, as will be appreciated by the context in which the elements are described.

As utilized in accordance with the present disclosure, the following terms, unless otherwise indicated, shall be understood to have the following meanings.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by anyone of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the inventive concept. This description should be read to include one or more and the singular also includes the plural unless it is obvious that it is meant otherwise.

Further, use of the term “plurality” is meant to convey “more than one” unless expressly stated to the contrary.

As used herein, qualifiers like “about,” “approximately,” and combinations and variations thereof, are intended to include not only the exact amount or value that they qualify, but also some slight deviations therefrom, which may be due to manufacturing tolerances, measurement error, wear and tear, stresses exerted on various parts, and combinations thereof, for example.

As used herein, the term “substantially” means that the subsequently described parameter, event, or circumstance completely occurs or that the subsequently described parameter, event, or circumstance occurs to a great extent or degree. For example, the term “substantially” means that the subsequently described parameter, event, or circumstance occurs at least 90% of the time, or at least 91%, or at least 92%, or at least 93%, or at least 94%, or at least 95%, or at least 96%, or at least 97%, or at least 98%, or at least 99%, of the time, or means that the dimension or measurement is within at least 90%, or at least 91%, or at least 92%, or at least 93%, or at least 94%, or at least 95%, or at least 96%, or at least 97%, or at least 98%, or at least 99%, of the referenced dimension or measurement.

The use of the term “at least one” or “one or more” will be understood to include one as well as any quantity more than one. In addition, the use of the phrase “at least one of X, V, and Z” will be understood to include X alone, V alone, and Z alone, as well as any combination of X, V, and Z.

The use of ordinal number terminology (i.e., “first”, “second”, “third”, “fourth”, etc.) is solely for the purpose of differentiating between two or more items and, unless explicitly stated otherwise, is not meant to imply any sequence or order or importance to one item over another or any order of addition.

Finally, as used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

The term “alkyl” refers to a saturated linear or branched hydrocarbon group of 1 to 50 carbons.

The term “alkylene” as used herein refers to an unsaturated, linear or branched hydrocarbon group of 1 to 50 carbon atoms with one or more carbon-carbon double bonds.

The term “alkylyne” as used herein refers to an unsaturated, linear or branched hydrocarbon group of 1 to 50 carbon atoms with one or more carbon-carbon triple bonds.

The term “aryl” refers to a mono- or polynuclear aromatic hydrocarbon group including carbocyclic and heterocyclic aromatic groups.

The term “hydrophobic” or “hydrophobically modified” as used herein refers to containing an alkyl, alkylene, alkylyne, and/or aryl group(s).

The term “monomer” refers to a molecule, typically having a molecular weight of less than or equal to about 1,000 Daltons, that chemically bonds during polymerization to one or more monomers of the same or different kind to form a polymer.

The term “polymer” refers to a macromolecular compound, typically having a molecular weight of from about 1,000 to about 500,000 Daltons, comprising one or more types of monomer residues (repeating units) connected by covalent chemical bonds. By this definition, the term “polymer” encompasses compounds wherein the number of monomer units may range from very few, which are more commonly called “oligomers,” to very many. The term “macromolecule” is used for individual molecules of high molecular weight and the term “polymer” can also be used to denote a substance composed of macromolecules. Non-limiting examples of polymers include homopolymers, and non-homopolymers such as copolymers, terpolymers, tetrapolymers, and higher analogues.

All percentages, ratios, and proportions used herein are based on a weight basis unless other specified.

The term “sample” as used herein will be understood to include any type of biological sample that may be utilized in accordance with the presently disclosed inventive concept(s). Examples of fluidic biological samples that may be utilized include, but are not limited to, whole blood or any portion thereof (i.e., plasma or serum), urine, saliva, sputum, cerebrospinal fluid (CSF), skin, intestinal fluid, intraperitoneal fluid, cystic fluid, sweat, interstitial fluid, extracellular fluid, tears, mucus, bladder wash, semen, fecal, pleural fluid, nasopharyngeal fluid, combinations thereof, and the like.

The term “patient” includes human and veterinary subjects. In certain embodiments, a patient is a mammal. In certain other embodiments, the patient is a human. “Mammal” for purposes of treatment refers to any animal classified as a mammal, including human, domestic and farm animals, nonhuman primates, and zoo, sports, or pet animals, such as dogs, horses, cats, cows, etc.

A “therapeutically effective amount” is any amount of any of the compositions and/or methods utilized in the course of practicing the inventive concepts provided herein that is sufficient to reverse, alleviate, inhibit the progress of, or prevent a disease, disorder or condition, or one or more symptoms thereof.

Turning now to the particular embodiments, the presently disclosed and claimed inventive concept(s) relate to improved biocompatible hydrogels, methods for making, and use of the same. In one non-limiting embodiment, the presently disclosed and claimed inventive concept(s) relate to a composition comprising a zwitterionic microgel (microscale hydrogel) loaded with at least one immobilized enzyme on the microgel.

### Zwitterionic Polymer and Microgel

### Zwitterionic Polymer

The zwitterionic polymer encompassed in any of the methods disclosed herein is now described in more detail. In any of the methods described herein, the zwitterionic polymer can comprise a crosslinked zwitterionic polymer having a crosslink to monomer ratio (molar) in a range of from about 1:50 to about 1:5, or from about 1:40 to about 1:5, or from about 1:30 to about 1:5, or from about 1:20 to about 1:5.

In certain embodiments, the zwitterionic polymer comprises a crosslinked zwitterionic polymer selected from crosslinked polysulfobetaine (SBMA), crosslinked polycarboxybetaine (pCB), crosslinked polyphosphobetain, and crosslinked polyphosphorylcholine. In one non-limiting embodiment, the zwitterionic polymer comprises a crosslinked polysulfobetaine methyl methacrylate (SBMA) polymer.

In certain embodiments, the zwitterionic polymer comprises a crosslinked zwitterionic polymer having covalent crosslinks, ionic crosslinks, or crosslinks formed by association of a portion of one zwitterionic polymer with another (zwitterionic fusion). In one non-limiting embodiment, the zwitterionic polymer comprises a crosslinked zwitterionic polymer having covalent crosslinks.

In certain embodiments, the zwitterionic polymer comprises a crosslinked zwitterionic polymer having degradable chemical crosslinks.

### Zwitterionic Microgel

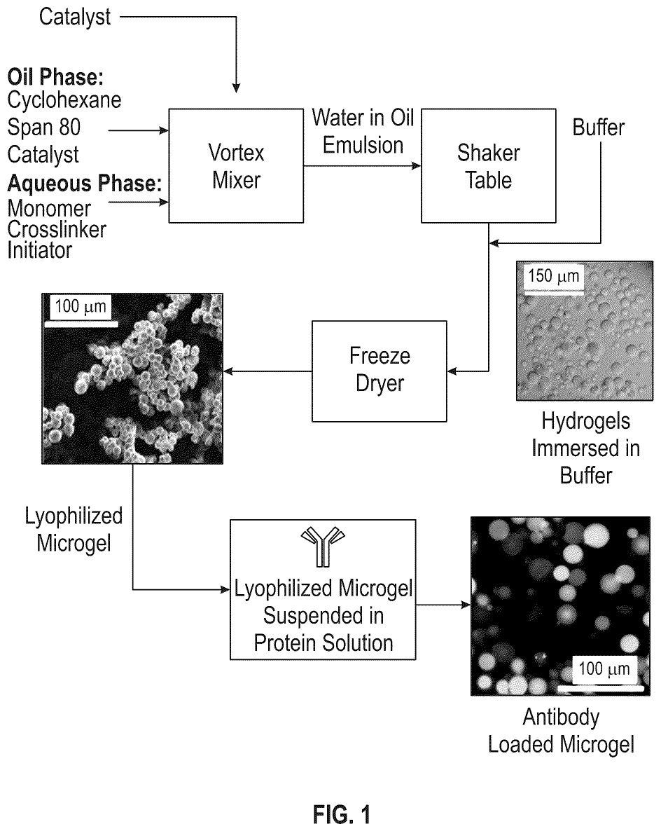

A zwitterionic microgel composition is prepared from physical processing of a crosslinked zwitterionic polymer or a crosslinked mixed charged polymer to provide a zwitterionic microgel composition comprising a plurality of crosslinked zwitterionic or a plurality of crosslinked mixed charged microgel units, respectively. A schematic drawing depicting the process for synthesizing the zwitterionic microgels is shown in FIG. 1.

In certain non-limiting embodiments, the zwitterionic microgel is lyophilized.

The prepared dried zwitterionic microgel has a diameter in a range of from about 1 micron to about 100 microns.

### Protein Loading

The zwitterionic microgel composition may further comprise a therapeutic agent, such as an active biomacromolecule. The active biomacromolecule can be an enzyme (e.g., α-chymotrypsin (ChT)), cytokines, globular protein (e.g., bovine serum albumin (BSA) or antibodies (e.g., Immunoglobin G)), or combinations thereof. In certain non-limiting embodiments, the protein of interest is ChT. The active biomacromolecule may be encapsulated or immobilized within the hydrogel. In certain embodiments, the at least one active biomacromolecule is chemically immobilized within the zwitterionic microgel.

A method of preparing a composition comprising a zwitterionic microgel loaded with at least one active biomacromolecule, the method comprising: (1) mixing at least one monomer, at least one crosslinker, and at least one initiator to form an aqueous phase; (2) mixing at least one oil soluble surfactant and at least one solvent to form an oil phase; (3) mixing the aqueous phase and the oil phase to form an emulsion; (4) reacting the emulsion with at least one catalyst to form a zwitterionic microgel composition; (5) lyophilizing the zwitterionic microgel composition; and (6) reacting the zwitterionic microgel composition with at least one active biomacromolecule by resuspending the zwitterionic microgel composition in a solution comprising the at least one active biomacromolecule to form a composition comprising a zwitterionic microgel loaded with at least one active biomacromolecule. In certain embodiments, the method further comprises a step (7) of lyophilizing the composition comprising a zwitterionic microgel loaded with at least one active biomacromolecule to produce a dried or freeze dried microgel.

In certain embodiments, the protein of interest is loaded in and/or immobilized on the zwitterionic microgel per weight of dried microgel in a range of from about 5 micrograms/milligrams to about 500 micrograms/milligrams, or from about 5 micrograms/milligrams to about 250 micrograms/milligrams, or from about 5 micrograms/milligrams to about 100 micrograms/milligrams, or from about 5 micrograms/milligrams to about 75 micrograms/milligrams, or from about 5 micrograms/milligrams to about 50 micrograms/milligrams, or from about 5 micrograms/milligrams to about 25 micrograms/milligrams. In one particular non-limiting embodiment, the protein of interest is loaded in and/or immobilized on the zwitterionic microgel per weight of dried microgel in a range of from about 5 micrograms/milligrams to about 50 micrograms/milligrams.

In certain non-limiting embodiments, the at least one monomer may be carboxybetaine methyl methacrylate or sulfo-betaine methyl methacrylate, or combinations thereof. In one particular non-limiting embodiment, the at least one monomer comprises carboxybetaine methyl methacrylate.

In certain embodiments, step (3) comprises mixing the aqueous phase and the oil phase using a vortex mixer in order to generate the emulsion. Step (4) may be performed at a temperature in a range of from about 4° C. to about 60° C., or from about 4° C. to about 25° C. and/or for a period in a range of from about 2 hours to about 4 hours. Step (5) may be performed at a temperature in a range of from about −80° C. to about −10° C. Step (6) may be performed at a temperature in a range of from about 0° C. to about 50° C., or from about 0° C. to about 40° C., or from about 4° C. to about 50° C., or from about 4° C. to about 40° C., or from about 20° C. to about 60° C., or from about 20° C. to about 25° C., or from about 20° C. to about 22° C. Step (6) may be performed at a time in a range of from about 2 hours to about 4 hours. Step (6) may be performed at a pH in a range of from about 3 to about 11.

Steps (5) and (6) may be repeated at least two times. In this way, cryo-protection of the active biomacromolecule is achieved during multiple cycles of freezing, lyophilization and rehydration without protein denaturation, as shown in the results below.

The zwitterionic microgel loaded with at least one active biomacromolecule is nonimmunogenic and does not cause immunogenic reaction, as shown in the results below.

### Examples

N-(3-(Dimethylamino)propyl)methacrylamide (DMAPAA), 3-propiolactone, (1-ethyl-3-(3-dimethylaminopropyl) carbodiimide hydrochloride (EDC), fluorescein isothiocyanate (FITC), and p-nitrophenyl acetate (NPA) were purchased form Sigma Aldrich. α-chymotrypsin (ChT) from bovine pancreas, N, N′-methylenebisacrylamide, tetra methyl ethylene diamine (TEMED), span 80, cyclohexane, and ammonium persulfate (APS) were purchased from Fisher Scientific. Bovine serum albumin (BSA), FITC-albumin conjugate, (2-(Methacryloyloxy) ethyl) dimethyl-(3-sulfopropyl) ammonium hydroxide (SBMA) and p-nitro phenyl caprylate were also purchased form Sigma Aldrich. All chemicals used were of analytical grade and used without any further purification.

### 1. Synthesis of the Carboxybetaine Methyl Methacrylate Monomers

Firstly, the carboxybetaine methyl methacrylate monomer was synthesized by reaction of DMAPAA and 3-propiolactone. For this purpose, 20.7 g DMAPMA was added to 450 ml anhydrous acetone. 3-propiolactone (9 ml, in 90 ml anhydrous acetone (10% v/v)) was slowly added to the DMAPAA mixture. The reaction was carried out at 25° C., in a sealed container (purged with nitrogen) for 6 hours, during which, the mixture was stirred using a magnetic stirrer. While the reactants were miscible in acetone, the product itself was insoluble in acetone. As such, the reaction product, a white precipitate, was separated from the solution by centrifugation. The product was washed in acetone twice to removed unreacted species and dried under vacuum for 48 hours before storage at 4° C. The extent of the reaction was gravimetrically evaluated to be 90%. The molecular structure of the product was confirmed using ¹H nuclear magnetic resonance (NMR).

### 2. Synthesis of the Microscale Polymer

### i. Synthesis of the Poly(Carboxy Betaine) Microscale Hydrogel

An inverse emulsion (aqueous phase dispersed in continuous oil phase) free radical polymerization reaction technique was used for the preparation of the microscale hydrogel. Monomer (CBMA), the crosslinker N, N′-Methylenebisacrylamide (bis-acrylamide), and the initiator, APS, were dissolved in PBS to constitute the aqueous phase. The concentration of APS in aqueous phase was set to 3 wt %. The crosslinker:monomer molar ratio was set to 1:16. The monomer concentration in the aqueous phase was set to 300 mg/ml. The oil phase consisted of 5 wt % Span 80 in cyclohexane. Span 80 is an oil soluble surfactant which facilitates the formation of a water-in-oil emulsion. The aqueous phase was added to the oil phase and a vortex mixer was used to mix the sample for 30 seconds to generate the emulsion. The ratio of oil to aqueous phase was fixed at 9:1 (v/v) to prevent phase inversion and coalescence of the dispersed phase. After emulsion formation, the catalyst, TEMED, was added to the emulsion (0.4 vol %). After 30 seconds of vortexing, the samples were placed on a shaker table to prevent settling of the reaction products (hydrogel beads) before the reaction was finished. The reaction was allowed to proceed at 25° C. (uncontrolled) for 4 hours. Later, the hydrogel beads were washed in cyclohexane to remove the Span 80 and washed with PBS, equilibrated in PBS for three days and subsequently washed using DI water. The washed hydrogel beads were thereafter frozen at −80° C. and lyophilized at −35° C. via low vacuum.

### ii. Synthesis of Polysulfobetaine Methacrylate (PSBMA)

Monomer (SBMA), the crosslinker N, N′-Methylenebisacrylamide (biz-acrylamide), and the initiator, APS, were dissolved in PBS to constitute the aqueous phase. The concentration of APS in aqueous phase was set to 2 wt %. The crosslinker: monomer ratio was varied between 1:50 to 1:5. The monomer concentration in the aqueous phase was varied for different samples. For the water content studies, the monomer concertation was set to 20 wt %. For the hydrogel diffusometry and protein loading/release samples, the monomer concentration was set to 25, 24, 20, 18, 16, and 10 wt %, for the different crosslinker: monomer ratios of 1:5, 1:10, 1:15, 1:20, 1:30, and 1:50 respectively. The oil phase consisted of 5 wt % Span 80 in cyclohexane. Span 80 is oil soluble which facilitated the formation of a water-in-oil emulsion. The aqueous phase was added drop-wise to the oil phase. A vortex mixer was used to generate the emulsion. The ratio of oil to aqueous phase was fixed at 9:1 (v/v) to prevent phase inversion and coalescence of dispersed phase. After emulsion formation, the catalyst, TEMED, was added to the emulsion (0.4 vol %). After 30 seconds of mixing, the samples were placed on a shaker table to prevent settling of the hydrogel beads before the reaction finished. The reaction was allowed to proceed for two hours. All reaction steps we carried out at 25° C.

### iii. Synthesis of Degradable Polycarboxybetaine (PCB) Microscale Hydrogel

Monomer, crosslinker (TTEGDA), and the initiator (APS) were dissolved in phosphate buffer saline (PBS) to constitute the aqueous phase. The molar crosslinker to monomer ratio was varied from 1:50 (low crosslinking density) to 1:3 (high crosslinked density), while the concentration of APS was held constant at 3 w/w %. The monomer concentration in the aqueous phase was set to 22 w/w %. The oil phase consisted of 1 w/w % span 80 in cyclohexane to assist the formation of a water-in-oil emulsion.

The CBMA monomer and polymerization initiator (ammonium persulfate) are water soluble: the reaction can only take place in the aqueous phase. However, the crosslinker can potentially dissolve (partition) in the oil phase, causing the crosslinker to not be available in the aqueous phase, and preventing formation of a hydrogel. To avoid this partition, an amount of the crosslinkerwas added to the oil phase to prevent migration of the crosslinker from aqueous to oil phase. The amount of crosslinker added to oil phase was based on the experimentally measured partitioning coefficient of the TTEGDA in both phases to prevent the crosslinker migration form one phase to another. The TTEGDA crosslinker was dissolved in cyclohexane at concentrations between 5 mg/ml (for low crosslinking) to 10 mg/ml (for high crosslinking) to prevent partitioning.

The aqueous phase was slowly added to the oil phase and mixed using a vortex mixer to generate a water-in-oil emulsion with a fixed oil to aqueous phase volume ratio of 9:1. After emulsion formation, the catalyst, TEMED, was added to the emulsion (1% v/v) in 6 aliquots while the sample was mixed. The reaction was allowed to proceed at room temperature for 6 hours on a shaker table to prevent settling of the microgel. After the reaction was completed, the samples were washed in cyclohexane to remove the Span 80 and subsequently washed with DI water. The oil and unreacted chemicals were separated from the microgel particles using centrifugation. The microgel was equilibrated in DI water for 3 days and later freeze-dried under vacuum (lyophilized) and stored at 4° C.

### 3. Enzyme Immobilization and/or Encapsulation

i. ChT Enzyme Immobilization within the Poly(Carboxy Betaine) Microscale Hydrogel

A post-fabrication protein loading/immobilization strategy was utilized. The lyophilized hydrogel was resuspended in a reaction mixture for the immobilization reaction to take place (1 ml reaction mixture for 40 mg hydrogel). The reaction mixture comprised 10 mg/ml ChT (enzyme) and 10 mg/ml EDC (zero-length crosslinker) dissolved in the reaction buffer. The reaction buffer comprised 100 mM phosphate buffer at pH 5, 0.2 mg/ml CaCl₂ (enzyme stabilizer), and 2 mg/ml NaCl. The reaction was carried out at 25° C. for 3 hours. At this point, the beads were washed with phosphate buffer (pH 5) several times to remove any unreacted ChT before equilibration in DI water. The enzyme immobilized hydrogels were lyophilized and later stored at 4° C.

ii. BSA Protein Encapsulation within the PSBMA Microscale Hydrogel

A post-fabrication protein loading strategy was utilized. The hydrogel beads were washed with DI water and frozen at −80° C. then placed in a freeze dryer. The dried beads were subsequently suspended in a concentrated protein solution (40 mg/ml BSA) for an extended period of time (3 to 5 days) at the 25° C. temperature.

iii. Antibody Encapsulation within the Degradable PCB Microscale Hydrogel

A post-fabrication Ab loading technique was utilized. The lyophilized microgel was resuspended in a 40 mg/ml Ab solution for 5 days at 4° C. in Tris HCl buffer (pH 8). Because the microgel is hydrophilic, the Ab solution soaked into the microgels to hydrate the polymer structure. The microgel beads were subsequently washed with PBS to remove any excess Ab solution.

### 4. Characterization of the Enzyme Immobilized Hydrogels

i. Confocal Microscopy and Preparation of Fluorescently Labeled ChT

Confocal microscopy was used for investigating the protein loading within the microscale hydrogel. A confocal laser microscope (Leica© DM E14) was used with 60× magnification lens. ChT was labeled using fluorescein isothiocyanate (FITC). For this purpose, ChT solution (2 mg/ml at pH 9 phosphate buffer) was prepared and kept at 4° C. 50 μl FITC solution (5 mg/ml in dimethyl sulfoxide) was thereafter slowly added to 1 ml of CT solution (5 μl aliquots). The subsequent reaction was carried out at 4° C. for 10 hours. The solution was thereafter diluted 10-fold and any unreacted FITC was separated using a 3.5 kDa dialysis membrane in exchange buffer (phosphate buffer with pH 5) for 12 hours. The fluorescently labeled ChT (FITC-ChT) was excited at 488 nm.

ii. Confocal Microscopy of PSBMA Microscale Hydrogel with Encapsulated BSA Protein

Confocal microscopy was used for investigating the protein loading inside the hydrogel beads. For this purpose, a confocal laser microscope (Leica© DM E14) was used with 60× magnification lens. The fluorescently labeled albumin (FITC-albumin conjugate, Sigma) was excited at 488 nm. The hydrogel beads were observed at 30 planes and the intensity of the fluorescent light emitted from each section was compared for evaluating the protein loading.

iii. Ultraviolet Spectra

The ultraviolet (UV) absorbances of the samples were acquired using a Beckman Coulter spectrophotometer (model DU 730). Blank and enzyme immobilized hydrogels were suspended in pH 7 phosphate buffer (15 mg hydrogel in 1 ml suspension) and free enzyme was dissolved in pH 7 phosphate buffer at 0.5 mg/ml

### 5. Equilibrium Water Content for PSBMA Microscale Hydrogel with Encapsulated BSA Protein

In order to determine the water content of the PSBMA microscale hydrogel, hydrogels were equilibrated in PBS or Deionized water for an extended amount of time (3 to 5 days) until their mass equilibrated at 25° C. The swollen hydrogels were weighed, freeze dried, and weighed again. For weighing the samples, water (or PBS) was poured out of the tube and the hydrogels were moved to a plastic weighing boat and the excess water (or PBS) was removed using delicate task wipers. The equilibrium water content (EWC) was calculated as follows:

$$EWC = \frac{mass of water}{total mass} \qquad ( Equation 1 )$$

### 6. NMR Diffusometry Analysis for PSBMA Microscale Hydrogel with BSA Protein

The diffusive properties of the gels were investigated using pulsed field gradient NMR diffusometry. A 400 MHz Bruker NMR spectroscope equipped with a gradient probe was used. The diffusion tests were carried out using stimulated echo sequence with 100 ms diffusion times. The hydrogel was prepared using the bulk polymerization technique. Hydrogels with different (crosslinker: monomer) ratios were prepared at their equilibrium water contents (previously measured) inside 5 mm NMR tubes. The solvent was prepared as a 9:1 H₂O/D₂O mixture. The self-diffusion of H₂O molecules was measured at 25° C. The reduced diffusivity of each hydrogel formulation was calculated as:

$${D}_{reduced} = \frac{{D}_{eff}}{{D}_{0}} \qquad ( Equation 2 )$$

In which D_{reduced} is the reduced diffusivity, D_{eff} is the measured, effective diffusivity in the hydrogel and D₀ is the diffusivity of the free H₂O/D₂O mixture. Furthermore, the tortuosity (c) and reduced diffusivity are related as follows:

$${D}_{reduced} = \frac{1}{τ} \qquad ( Equation 3 )$$

### 7. Activity Assay for the Free and Immobilized Enzymes

i. Activity Assay for the Free Enzyme

ChT esterolytic enzymatic activity was measured using a nitrophenyl acetate (NPA) activity assay. Enzyme solutions were prepared at a concentration of 0.25 mg/ml in 100 mM phosphate buffer at the desired pH condition. 5 μl of an 8 mM NPA solution was thereafter added to 100 μl of ChT sample. The samples were then stored at a temperature of from 20° C. to 70° C. for 30 minutes. The absorbance of the samples was measured at 410 nm using a Packard SpectraCount plate reader. The activity measurements were normalized between the highest measured absorbance and the absorbance of buffer+NPA. The experiments were carried out in triplicate.

ii. Activity Assay for Immobilized Enzyme

First, 40 mg of solid enzyme-immobilized hydrogel was hydrated and suspended in 1500 μl phosphate buffer at the desired pH. Aliquots of the suspension (100 μl) were pipetted inside wells of a 96 plate. 5 μl of an 8 mM solution of NPA was thereafter added to each well. The absorbance of the samples was measured at 410 nm, and activity measurements were normalized between the highest measured absorbance and the absorbance of the pCB hydrogel samples against a no ChT (blank hydrogel) sample. The experiments were carried out in triplicate.

### 8. Protein Release and Bioactivity of the Released Proteins

i. Measurement of Protein Release for PSBMA Microscale Hydrogel with Immobilized BSA Protein

The hydrogel beads were loaded with fluorescently labelled BSA at 25° C. The hydrogels were thereafter immersed in PBS solution and the amount of protein released form the gel was determined using florescence spectroscopy. The volume ratio of the PBS buffer to the hydrogel beads was set to 30. The PBS was periodically replaced with fresh buffer every 24 hours to preserve the sink conditions. The excitation and emission wavelengths were set to 485 and 535 nm, respectively. Solutions with different known concentrations of fluorescently labelled BSA in buffer were prepared and the fluorescence intensity of each sample was measured to prepare a standard plot.

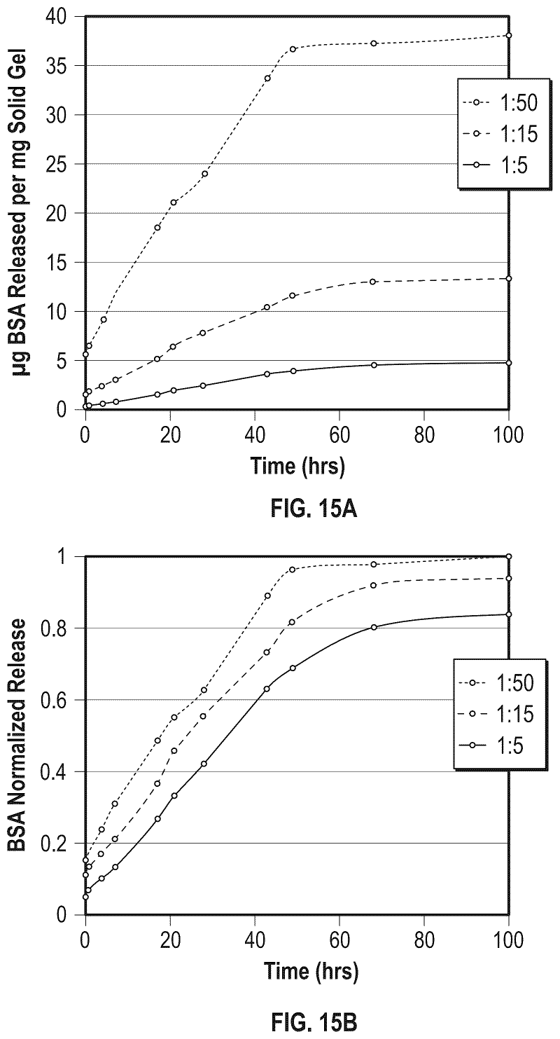

In another example, the release was carried out at 37° C. in PBS. As shown in FIGS. 15A-15B, the lower crosslinked hydrogel released more protein than the higher crosslinked hydrogels at all times measured. The plots further show that BSA was initially released following a zero-order relationship.

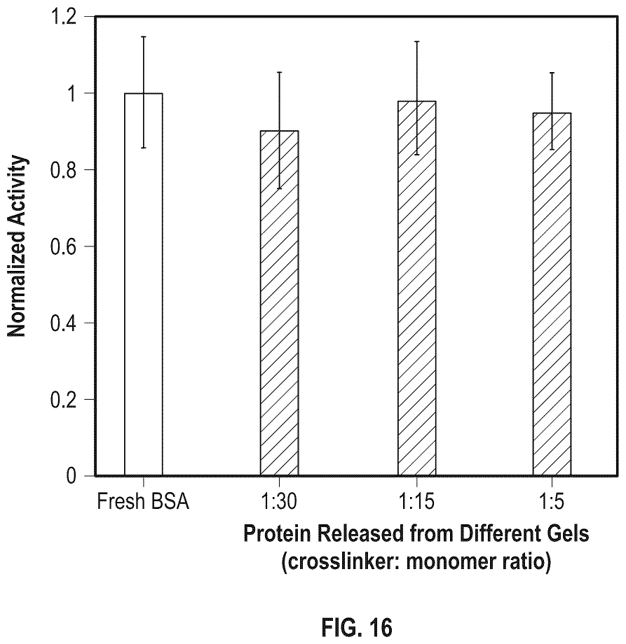

The enzymatic activities of the released proteins (FIG. 16) indicates that for crosslinker: monomer ratios of 1:30 to 1:5, no significant changes in activity were observed. Given the activity of the BSA, the protein molecules were not denatured. The ability of the zwitterionic hydrogel beads to load and release BSA without loss of esterolytic activity is significant when compared to similar protein encapsulation technologies. For example, studies on BSA encapsulation in PLGA microspheres indicated that up to 50% of the BSA aggregated or was bound to the polymer in such a way that led to a substantial loss of activity during fabrication. See Crotts, G.; Park, T. G., Stability and release of bovine serum albumin encapsulated within poly(d,l-lactide-co-glycolide) microparticles. Journal of Controlled Release 1997, 44 (2), 123-134.

ii. Bioactivity of Released Proteins for PSBMA Microscale Hydrogel with Encapsulated BSA Protein

The activity test was performed to determine if the proteins, once released from the hydrogels, retain their native conformation and enzymatic activity. The BSA esterolytic activity was measured, therefore, using a p-nitro phenyl caprylate activity assay. Samples of fresh BSA and BSA released from the hydrogels were prepared at 0.250 mg/ml concentration. The bicinchoninic acid assay (BCA) was used to measure the released BSA concentration, thereby completing the assay according to protocol. 3.63 μl of a 6 mM solution of caprylate was thereafter added to 100 μl of the BSA sample. The resulting samples were stored at 37° C. for 4 hours. The absorbance of the samples was measured at 410 nm using a Packard SpectraCount plate reader. Furthermore, the protein activity was normalized by protein concentration. The experiments were carried out in triplicate.

iii. Release of IgG and IgG Activity

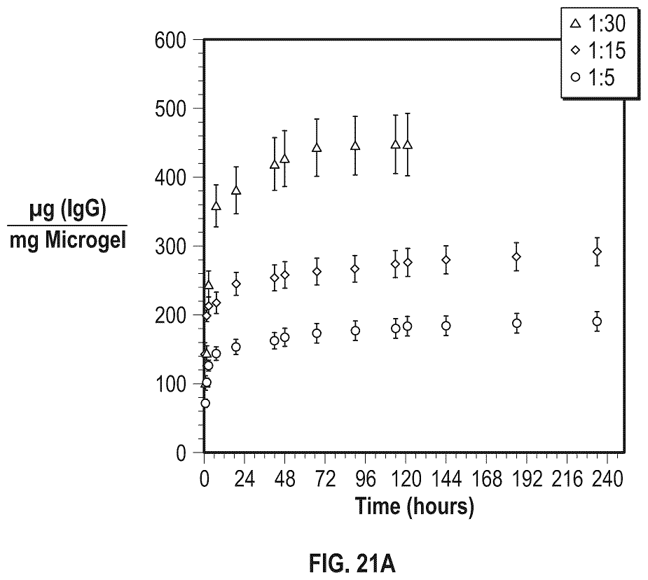

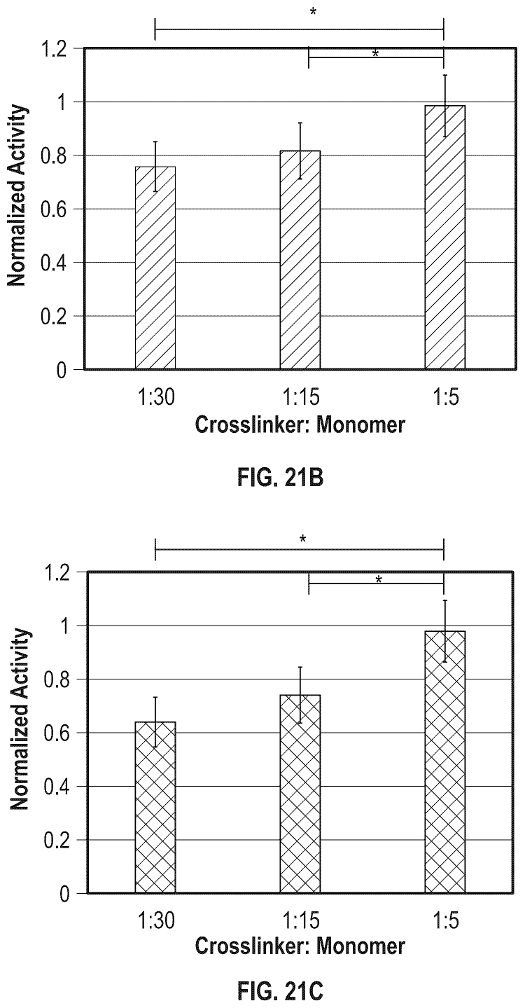

The FITC-IgG loaded microgels of different crosslinking densities were dispersed in PBS buffer and stored at 40° C. (10 mg of microgel was loaded and immersed in 2 ml PBS). The PBS buffer was replaced with fresh PBS after each sample was taken, and the removed sample was stored at 4° C. FITC-IgG concentrations were evaluated using a fluorescence plate reader and by comparing to a previously made calibration curve for the free FITC-IgG fluorescence intensity (samples in triplicates). The release of Ab from microgels is shown in FIG. 21.

The ability of the released antibodies to bind with Ab receptors was studied with a separate ELISA. Released IgG (not labeled) solution from the microgels were diluted to known concentrations and evaluated using an anti-Ab pre-coated ELISA kit for total human IgG.

The results indicated that the total amount of Ab that can be released from the microgels decreased with increasing the crosslinking density. The hydrogel swelling ratios for the 1:30, 1:15 and 1:5 microgels were 38.1, 20.4 and 9.5 accordingly. The total loaded IgG loadings for these microgels are 448, 291, and 191 (g/mg). The resulted indicated that the swelling ratio and the IgG loading had a direct relationship.

The post-release functionality of Ab from the sustained delivery matrix is a critical factor because concentrated Ab solution is susceptible to aggregation and denaturation at body temperature. The results indicate that the Abs released from the 1:5 microgel completely retained their function. This was true for the early stage released Abs (first 18 hours of release) and later stage of release (18 to 120 hours). Such results indicate the benefit of using zwitterionic microgels. For the lower crosslinking density of 1:30, the majority of the released Abs are active but the activity is significantly lower. For the early state released Abs 78% and for later stage released 67% of Ab molecule were active.

### 9. Half-Life Measurements

The static (no substrate during the incubation) half-life of free and immobilized enzymes was measured at different pH levels (i.e., pH of 4-9) and at different temperatures (i.e., 30-60° C.). For each study, seven samples were stored at the desired temperature and pH levels and subsequently removed at different time intervals and stored at 4° C. When all 7 sample were collected, the activity of all the samples was measured at T=40° C. and at a pH of 7.5. All the measured activities were normalized based on the activity at time zero. The decay of the activity (U) was calculated to fit the half-life of the enzyme:

$$U = {U}_{0} * {( \frac{1}{2} )}^{\frac{t}{{t}_{\frac{1}{2}}}} \qquad ( Equation 4 )$$

where U₀ is the activity at time zero; t is time; and t_{1/2} is the half-life.

### 10. Protein Stability for PSBMA Microscale Hydrogel with Encapsulated BSA Protein

In order to test for temperature stabilization, the samples (protein solutions and protein-loaded hydrogels) were incubated at 70° C. for differing periods of time to measure protein susceptibility to aggregation. After incubation, the protein from the hydrogels were released into PBS buffer. The samples of released protein were analyzed using native poly acrylamide gel electrophoresis (N-PAGE) to compare the amount of protein monomer lost due to aggregation. Direct comparison was carried out for protein in solution as well as protein inside the hydrogel at equivalent protein concentrations. Samples were loaded onto an 8 wt % polyacrylamide gel and were run at 200 V. A Bio-Rad Tetra Cell mini gel electrophoresis apparatus was utilized for this purpose. Gels were stained with Coomassie G-250 before imaging. The relative band intensities were compared to determine the amount of protein monomer present in each sample using UVP imaging system software. Furthermore, the BSA esterolytic enzymatic activity of the thermal stability samples was also measured. Experiments were carried out in triplicates.

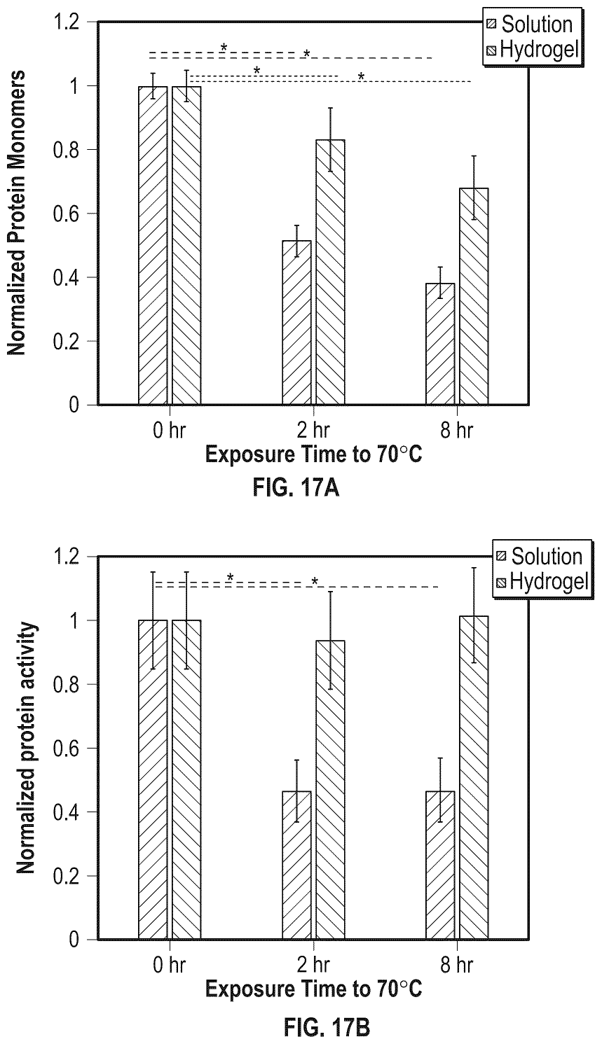

As shown in FIG. 17A, while the BSA tended to aggregate in solution as well as inside the hydrogels (at 70° C.), the aggregation rate inside the hydrogel was significantly slower. For example, after 8 hours the quantity of monomers in solution was less than 40% while for the hydrogel it was around 70%. This lower aggregation rate is due to the smaller diffusion coefficients inside the hydrogel (compared to free water, which slows down molecular motion) and is in accordance with the zwitterions' ability to affect the activation energy gaps in protein aggregation, as previously reported. See, Goldberg, M. E.; Expert-Bezangon, N.; Vuillard, L.; Rabilloud, T., Non-detergent sulphobetaines: A new class of molecules that facilitate in vitro protein renaturation. Folding and Design 1996, 1 (1), 21-27. The difference between the enzymatic activity of BSA molecules after exposure to high temperature is illustrated in FIG. 17B, which shows that while the BSA in the solution lost more than half of its enzymatic activity (due to significant tertiary structure changes), the proteins released from the zwitterionic hydrogel retained all of their enzymatic activity. The ability of the zwitterionic hydrogel to improve protein stability results in sustained protein delivery as well as improved protein immobilization inside the hydrogels.

### 11. In Vitro Cell Viability Assay

i. PSBMA Microscale Hydrogel with Encapsulated BSA Protein

First, zwitterionic polymer pSB was synthesized using free radical polymerization. The reaction was carried out at 25° C. in a buffered solution with a pH of 7.6 (PBS). The SBMA monomer was dissolved in PBS at 200 mg/ml concentration. A 10 vol % of APS solution (10 wt % APS in PBS) and 0.4 vol % TEMED was used as the initiator and catalyst, respectively. The reaction proceeded for 4 hours on a shaker. The pSB was then purified using a 3.5 kDa dialysis membrane. 1H NMR was used to confirm the reaction and to evaluate the average molecular weight. End group analysis, in which the end groups and the repeating monomer groups were identified and accurate integration was used to determine the average molecular weight of the produced proteins. The evaluation of the molecular weight using 1H NMR was only used for the polymer in solution and was not used for the crosslinked hydrogel beads.

To determine if pSB and hydrogels are cytotoxic, a cell viability assay was used to measure pSB and hydrogel cytotoxicity in vitro. Mice fibroblast cells (NIH 3T3) were seeded at 2×105 cells/well in DMEM supplemented with 10 vol % calf serum approximately 24 hours before beginning the cell viability assay. Varying concentrations of pSB (0-3 mg/ml) were added to the wells and treated for 24 hours. The Cell Titer Blue cell viability assay (Promega) was performed to evaluate the cell viability by measuring fluorescence intensity. In the case of gel beads, hydrogels were added to the wells at different weight to volume concentrations to the point that a layer of hydrogel beads covered the seeded cells. After treatment for 24-hours, the hydrogel beads were removed, by rinsing the cells with PBS and aspirating. Cell viability was measured for 5 replicates for each sample using the Cell Titer Blue cell viability assay. The cell viability was calculated using:

$$( Equation 5 )$$

$$Cell viability := \frac{{Fluorescence}_{sample} - {Fluorescence}_{Isopropanol Treated Cells}}{{Fluorescence}_{Untreated Cells} - {Fluorescence}_{Is opropanol Treated Cells}} \times 100 \%$$

ii. Biodegradable PCB Microgel with Encapsulated Antibody

To evaluate microgel hydrolysis-based degradation, microgels were dispersed in PBS and stored at 40° C. (with no agitation or stirring). For each crosslinking density, multiple samples were stored and studied at different time intervals. At each time interval, a sample was removed and washed using DI water to remove degraded water-soluble products. Subsequently, the sample was dried and weighed and the change of mass of the microgels for the sample was used to determine microgel degradation (studies were carried out in in triplicate). The intervals between each sampling were varied between 1-4 days.

Mouse fibroblast cells (NIH 3T3) were seeded in a 96-well plate at 2.5×10⁴ cells/well in DMEM supplemented with 10% v/v calf serum approximately 24 hours before the cell viability assay. Varying concentrations of the microgel (0.05%-5% v/v) were added to the wells and treated for 24 hours. The microgel beads were then removed by rinsing the cells with PBS. The Cell Titer Blue cell viability assay (Promega) was performed (6 replications) to evaluate the cell viability by measuring fluorescence intensity. In addition to the microgels, the degradation products of the microgels (soluble polymers) were separated and evaluated for cytotoxicity.

iii. In Vitro Cytotoxicity and Immunogenicity of the Biodegradable PCB Microgel with Encapsulated Antibody

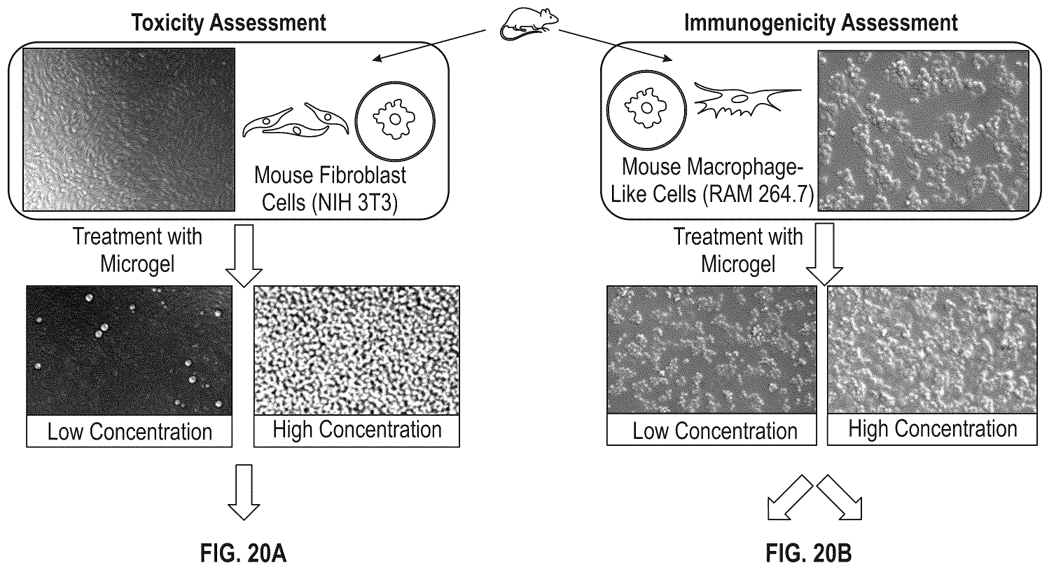

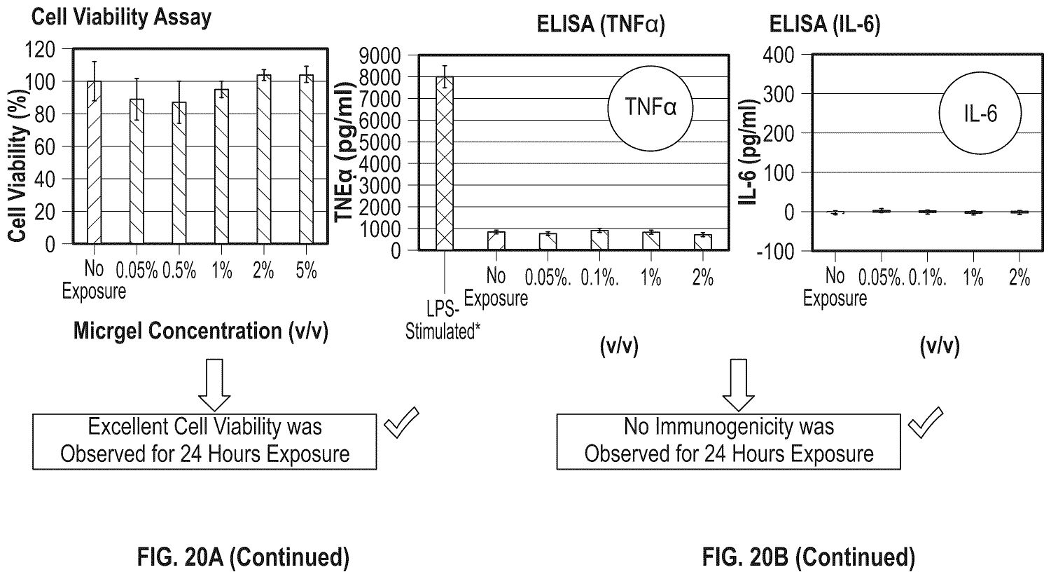

Cell compatibility is a requirement for the soft delivery of biomolecules. Fibroblasts are abundant in SC tissue. The viability of such cells when exposed to the microgels was evaluated, with the results depicted in FIG. 20. To perform this test, microgels were added to cell culture wells and cell viability was evaluated after 24 hours of exposure. The cells exposed to microgels showed 100% cell viability (no statistically significant difference between exposed and control cells). Additionally, the cells exposed to the degradation products of the microgels at concentrations of up to 2 mg/ml showed 100% cell viability.

The immunogenic reaction of mice macrophage cells to the microgels was studied by quantifying cytokine secretion from macrophage cells in vitro. RAW 264.7 cells were seeded at 3×10⁵ cells/well in DMEM at 37° C. The cells were treated with microgels at different concentrations (4 replicates) for 24 hours before cell media were sampled. Secretion of interleukin 6 (IL-6) and tumor necrosis factor-alpha (TNFα) were studied using two separate pre-coated enzyme-linked immunosorbent assay (ELISA) kit. After 24 hours exposure to the microgel, the cells did not secrete any measurable amount of IL-6. For TNF-alpha, the cells secreted approximately 500 pg/ml TNF-aplha with or without exposure to the microgels. This amount of TNF-alpha is significantly smaller than secretion caused by an immunogenic reaction to an endotoxin molecule such as lipopolysaccharides which can be approximately 8000 pg/ml or higher. See, Agbanoma, G.; Li, C.; Ennis, D.; Palfreeman, A. C.; Williams, L. M.; Brennan, F. M., Production ofTNF-α in Macrophages Activated by T Cells, Compared with Lipopolysaccharide, Uses Distinct IL-10-Dependent Regulatory Mechanism. The Journal of Immunology 2012, 188 (3), 1307-1317.

### 12. Enzyme Immobilized Zwitterionic pCB Hydrogel

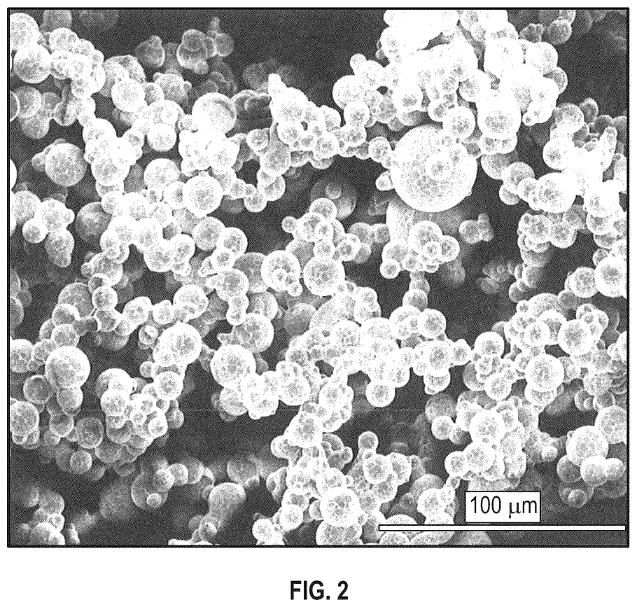

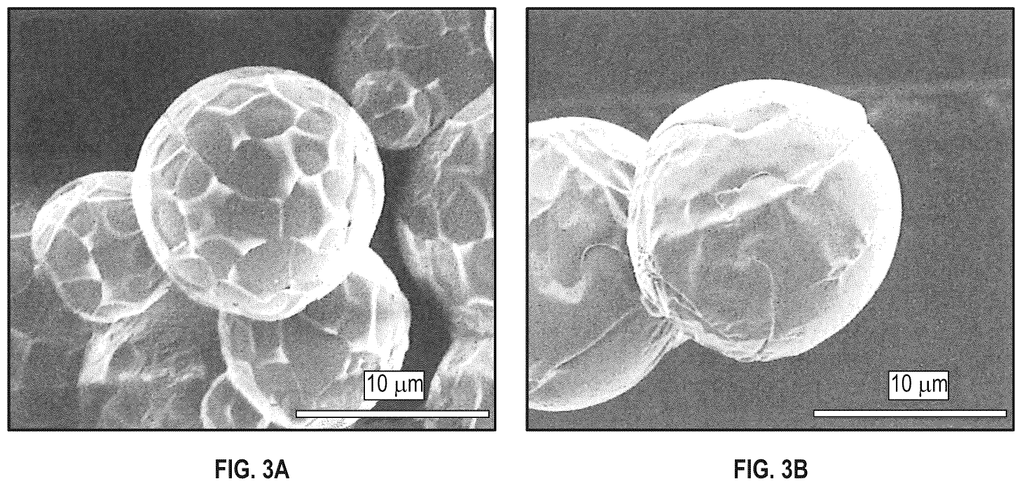

SEM micrographs of the lyophilized (freeze dried) pCB microscale hydrogels (FIG. 2) show that most of the hydrogel beads are within 5-17 microns in size and take the form of separate spherical particles. The crosslinker:monomer molar ratio for the depicted sample was 1:16. FIG. 3 compares the surface structure of the pCB hydrogel and the enzyme immobilized pCB hydrogel.

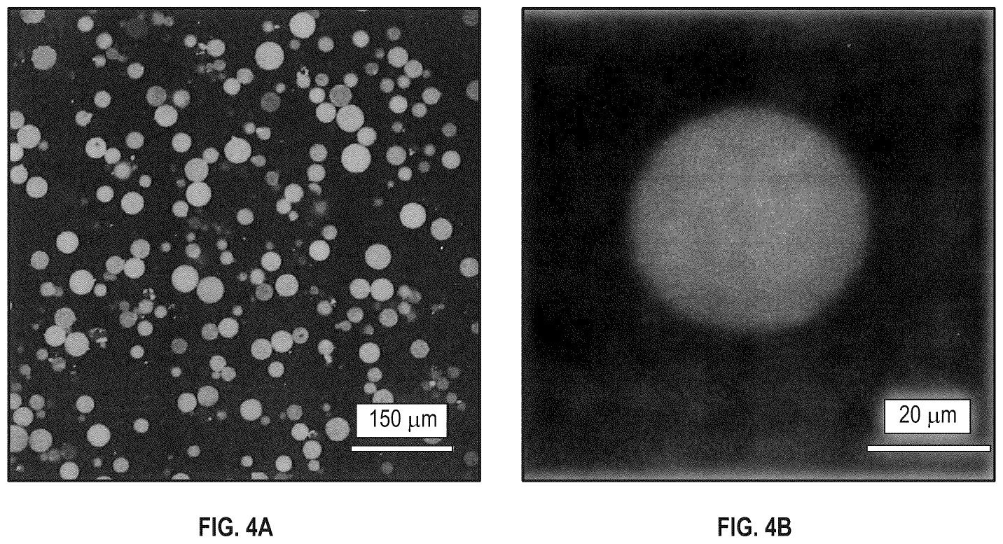

Furthermore, confocal microscopy shows in FIG. 4 the fluorescently tagged ChT within the microscale hydrogels. The enzyme immobilized hydrogel was suspended in buffer for 5 days for the unreacted enzyme to release from the hydrogel. Several confocal microscopy studies were carried out to investigate the distribution of the enzyme inside the hydrogel at multiple planes. FIG. 4B shows the averaged projection of a series of optical sections at different depths of the bead. FIG. 4B demonstrates that the protein molecules were homogenously immobilized inside the hydrogel; not only surface bound.

The entrance of protein and EDC crosslinker into the dried hydrogel occurred as a result of capillary and osmotic pressure that forms when the lyophilized hydrogel beads are immersed in a protein and EDC crosslinker solution. Such a loading strategy can be applied if the hydrogels are small enough for the capillary/osmotic loading to take place in a reasonable time. By comparison of the hydrogels in FIGS. 3 and 4, while dried hydrogels average diameter was 13 μm the same swollen hydrogels have an average diameter of 35 m.

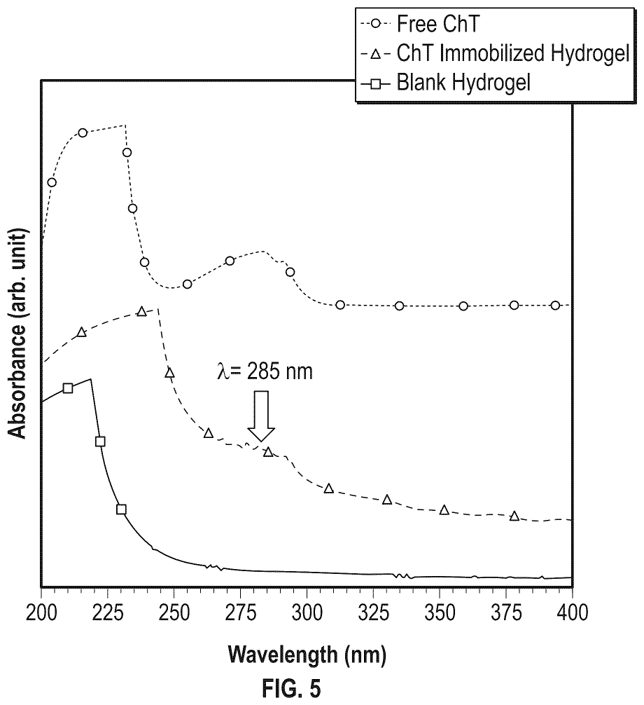

To further characterize the hydrogels, UV spectra of the enzyme immobilized hydrogel was compared to blank hydrogel and free enzyme (FIG. 5A). The UV spectra of the ChT immobilized hydrogels resembles the superposition of the blank hydrogel and the free ChT. This indicates that the ChT is immobilized inside the hydrogel.

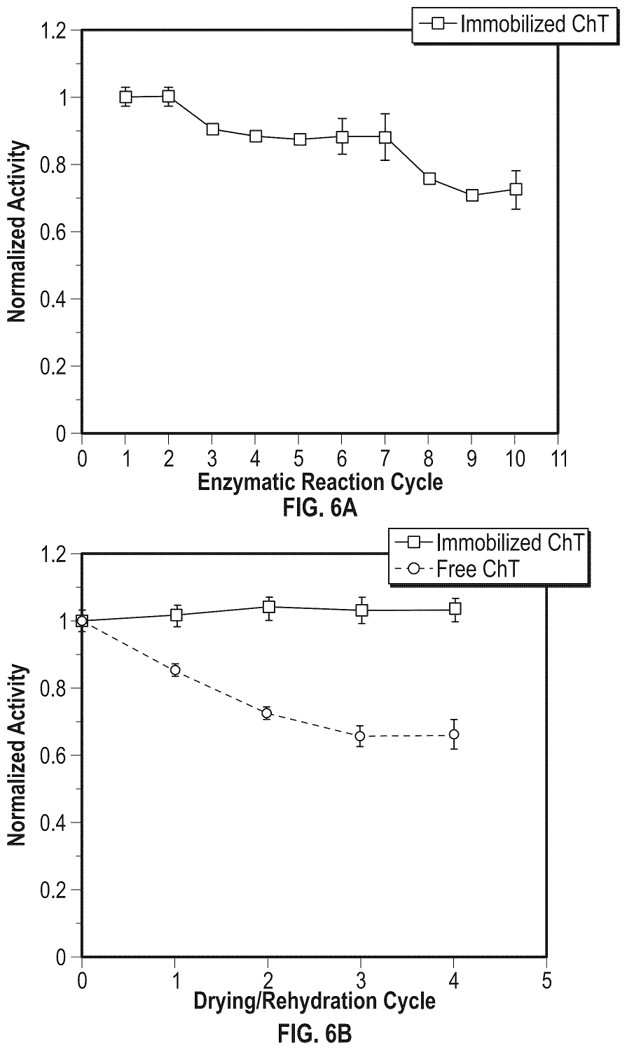

Enzyme reusability is an important functionality of an immobilized enzyme and is shown in FIG. 6A. Immobilized enzyme retained 72% of its initial activity after 10 reuses. As such, the biomolecule, in this example ChT, was loaded after the microgel was synthesized. This methodology prevented unwanted chemical reactions and biomolecule aggregation and denaturing to occur if the enzyme is costly or is being used in sensitive applications in which denatured enzymes might show immunogenic reactions or lose their bio-affinity. The discussed method has significant advantages. Furthermore, after the immobilization reaction the hydrogel beads were again lyophilized. The lyophilized enzyme immobilized hydrogels retained their enzymatic activity for four cycles of freeze drying/rehydration (FIG. 6B). The hydrogel can be dried for storage and rehydrated, therefore, prior to use without any need for a cryoprotectant.

i. Biodegradable pCB Microgels with Encapsulated Antibody

Most of the microgel beads formed were within 5-35 microns in diameter. The average diameter of the dried microgels decreased from 25.6 m for the less crosslinked microgel to 11.4 for the highly crosslinked microgel. The surface of the 1:5 crosslinked microgels was rougher than that of the 1:15 crosslinked microgels. This shrinkage can be attributed to the stress induced by crosslinker molecules deforming the polymer strands. Furthermore, the microgels with the lowest crosslinking ratio (1:30) had more structural imperfections. Although distinct particles were observed, most particles were agglomerated together.

The microgels were extremely hydrophilic and swelled with high water contents at all crosslinking densities. The microgels had a water content up to 97.4% and a swelling ratio of approximately 38 for the low crosslinked microgels (1:30) which is higher than several previously studied pCB hydrogels. See Chien, H.-W.; Yu, J.; Li, S. T.; Chen, H.-Y.; Tsai, W.-B., An in situ poly (carboxybetaine) hydrogel for tissue engineering applications. Biomaterials science 2017, 5 (2), 322-330. See Yang, W.; Bai, T.; Carr, L. R.; Keefe, A. J.; Xu, J.; Xue, H.; Irvin, C. A.; Chen, S.; Wang, J.; Jiang, S., The effect of lightly crosslinked poly(carboxybetaine) hydrogel coating on the performance of sensors in whole blood. Biomaterials 2012, 33 (32), 7945-7951. For the high crosslinked microgel (1:3), the water content was 88.0%. By comparison, pCB hydrogels crosslinked with methacrylate were previously shown to a have a lower water content of 70%. See, Carr, L. R.; Xue, H.; Jiang, S., Functionalizable and nonfouling zwitterionic carboxybetaine hydrogels with a carboxybetaine dimethacrylate crosslinker. Biomaterials 2011, 32 (4), 961-968. The high water content of the TTEGDA-crosslinked microgel indicates that the flexibility of the crosslinker did not prevent hydration even at high crosslinking densities.

The FTIR spectra of the monomer was compared to microgels with different crosslinking densities. The monomer had a peak at 1670 cm⁻¹ for the C═C bond which disappeared after polymerization. The 1080 cm⁻¹ peak corresponding to the C—O—C bond was associated with the TTEGDA crosslinker. The intensity of the FTIR peak increased at higher crosslinking densities, indicating that crosslinking densities of the samples were correctly controlled. The NR₄⁺ bond was observed at 3250 cm-1. Furthermore, peaks for the asymmetric and symmetric —COO— stretching were observed at 1590 cm-1 and 1370 cm-1, respectively, indicating a negative charge due to unprotonated carboxylic acid for all crosslinking densities. Similarly, the positively charged ammonium was observed at 2800-3000 cm-1, confirming the zwitterionic nature of the microgels.

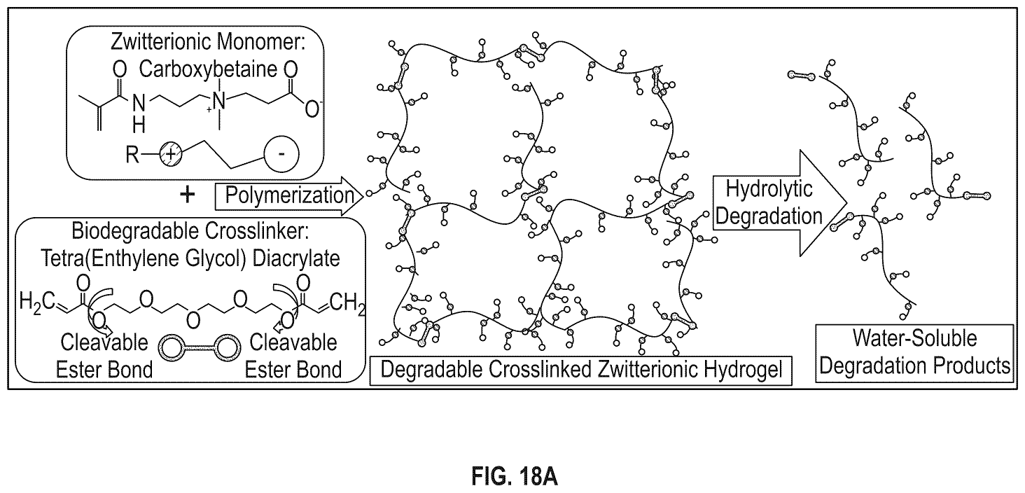

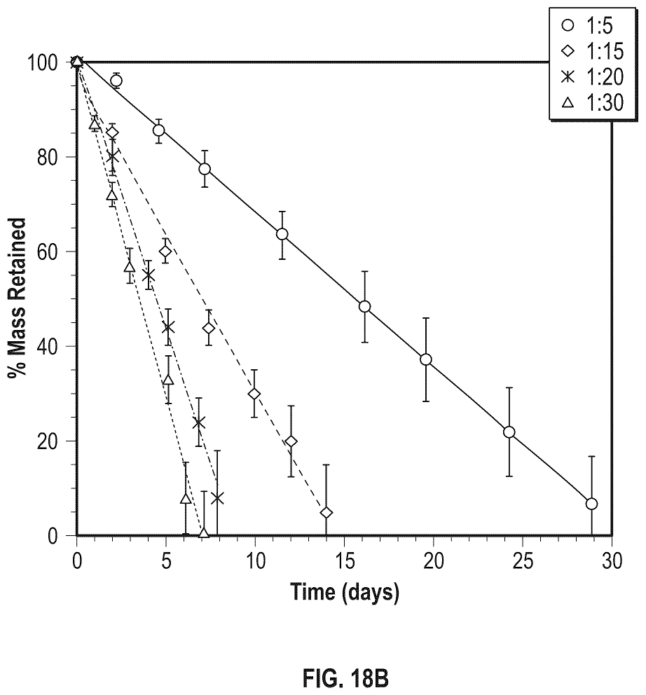

Degradation of the microgel is illustrated in FIG. 18. In aqueous solution, the cleavable ester bond in TTEGDA can hydrolytically degrade and form water-soluble pCB polymers. The hydrolytic degradation of the different crosslinking densities was studied. Microgel degradation is a function of crosslinking density. The low crosslinked (1:30) microgels fully degraded in approximately 7 days; the high crosslinked (1:5) microgels fully degraded in approximately 30 days.

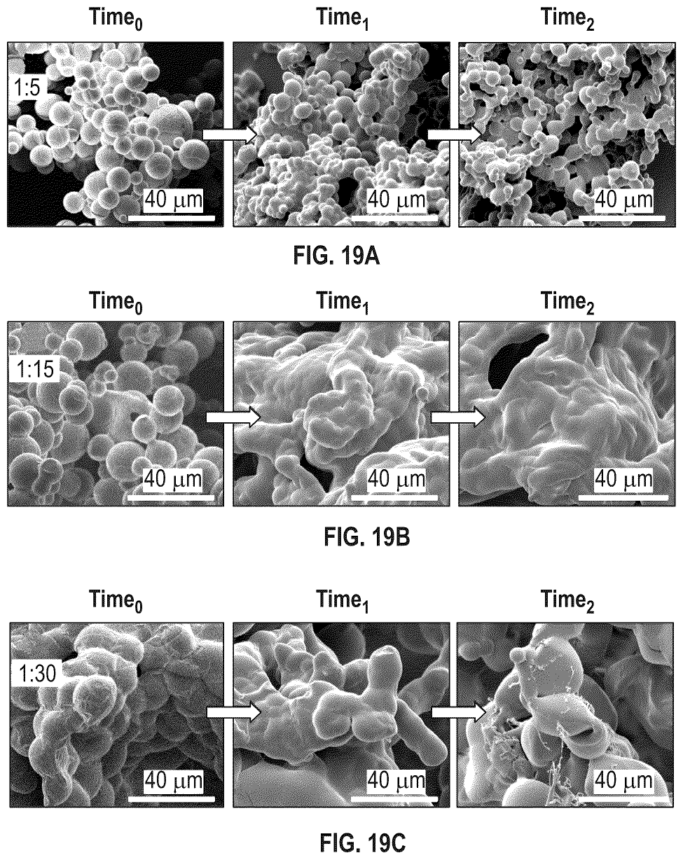

The shape and structure of the microgel during degradation can be seen in FIG. 19. For the high crosslinking density, the particles kept their overall shape during degradation, but their surfaces became increasingly smoother. The less crosslinked microgel particles tended to aggregate during the degradation process. Microgel aggregation also occurred during degradation. The microgel aggregation (as opposed to agglomeration) is dependent not only on the adhesion forces between the particle, but also on the mechanical strength of the microgel. For the low crosslinked microgels, the weakened mechanical strength during degradation promotes the particle aggregation. The adhesion forces between the microgels can be caused by the interactions between the zwitterionic polymer chains or by interaction between the TTEGDA moieties.

Confocal fluorescence microscopy studies of the microgels loaded with FITC-IgG showed that all beads are loaded with the Ab. This study demonstrated that the FITC-IgG molecules were homogenously loaded within the microgel and were not only surface bound.

### 13. Enzymatic Activity of Immobilized ChT at Different Temperatures and pH

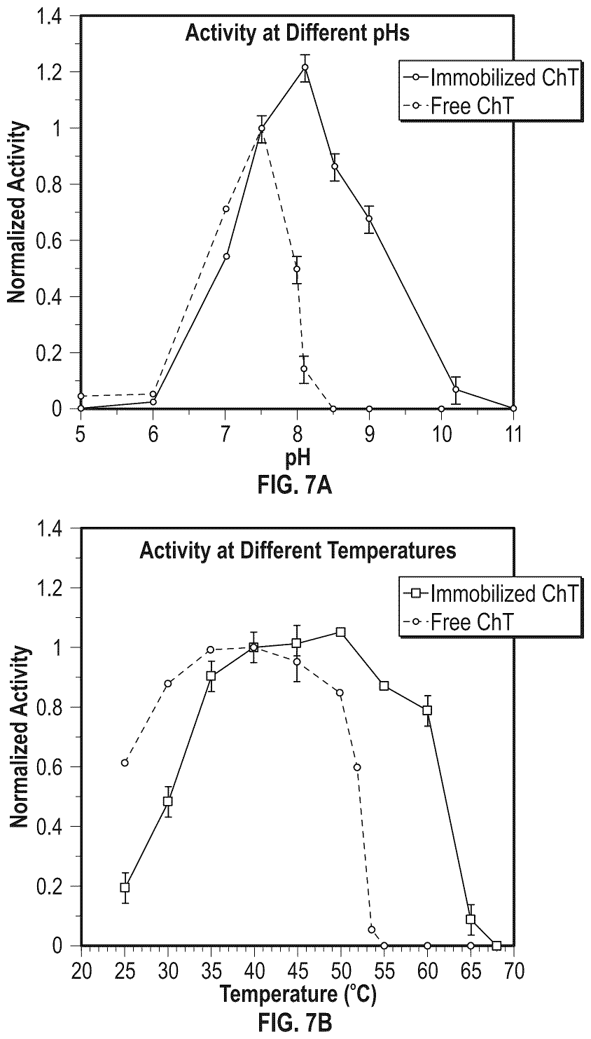

Enzymatic activity of ChT is highly temperature and pH dependent and has a bell-shaped curve. The activity of immobilized and free enzyme was compared and the results are shown in FIG. 7. In particular, as shown in FIG. 7A, free ChT activity sharply decreased above a pH of 8. In contrast, the immobilized ChT (i-ChT) is significantly more active at a higher pH than that of physiological pH. The i-ChT is most active at a pH of 8.5 and is partially active up to a pH of 10.2. As such, immobilized enzymes or biomolecules in the hydrogels disclosed herein, exhibit useful properties and activities at non-physiological conditions.

The enhanced activity of i-ChT at basic pHs is due, at least in part, to the free enzyme reaching or exceeding its isoelectric point (pl 8.50) at a pH of 8.0 or greater. The lack of electrostatic charge allows direct interactions between ChT, leading to structural changes, and protein aggregation. In contrast, the i-ChT retains activity as any direct interactions between protein molecules is limited since the protein is immobilized. As mentioned previously, at pH of 8-10 the loss of activity is caused by physical aggregation and denaturing (thermophysical deactivation). In contrast, at pH 10-11 the α-amino group of isoleucine 16 becomes deprotonated causing the salt bridge between isoleucine 16 and asparagine 194, critical for the enzyme active conformation, to break (chemical deactivation). As a result, the ChT structure changes to a chymotrypsinogen-like structure (proenzyme). i-ChT is active up to pH 10.2, which shows that immobilization prevents thermophysical deactivation.

In acidic environments, free ChT was more active than the i-ChT since the free ChT is capable of forming dimeric chymotrypsin. Although activity is relatively small, such activity is still available to cleave NPA at a pH as low as 3.7. The immobilized enzymes are not as flexible and able to form active dimers, with results of reduced activity of i-ChT in acidic environments.

As indicated in FIG. 7b, free ChT has its highest activity at 40° C. At higher temperatures, the activity sharply decreased, and finally at temperatures above 54° C. free ChT lost its activity. This is aligned with previously reported melting point of ChT at 53.4° C. See, Kumar, A. and P. Venkatesu, Overview of the stability of α-chymotrypsin in different solvent media. Chemical Reviews, 2012. 112(7): p. 4283-4307. In contrast, activity of i-ChT increased by increasing the temperature to 52° C. and only decreased gradually thereafter. Furthermore, i-ChT retained its activity up to 65° C. which indicated a significant increase in the thermal stability and thermal activity of the immobilized enzyme. At low temperatures, i-ChT showed normalized enzymatic activity below that of the free ChT, which is due to the increased diffusion limitations caused by the low permeability of hydrogel at low temperatures resulting in a diffusion-controlled reaction rate.

### 14. BSA Laden Zwitterionic PSBMA Hydrogel

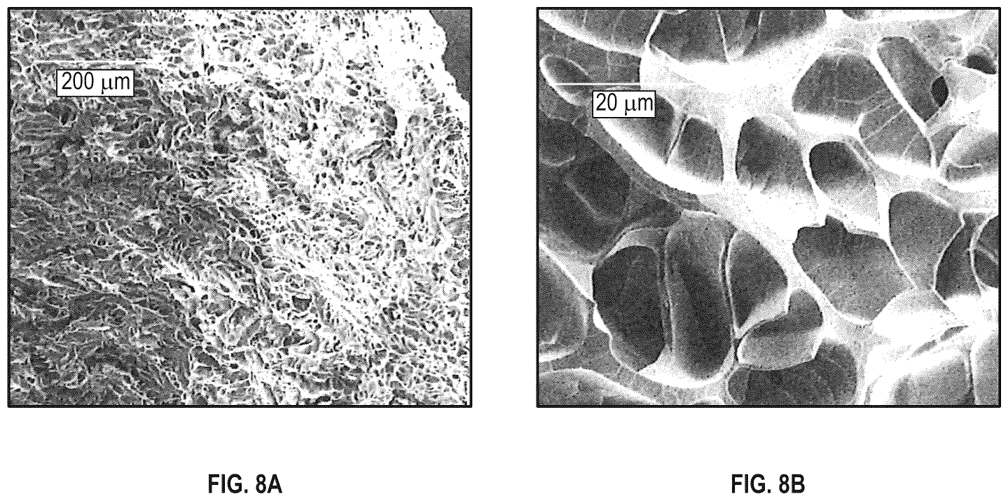

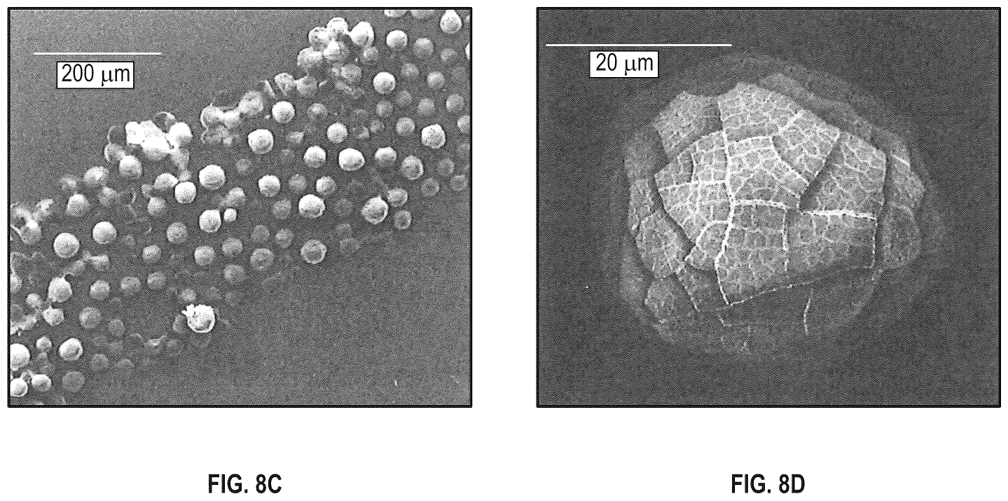

SEM micrographs of the lyophilized (freeze dried) hydrogel beads (FIG. 8A-8D) show that most of the hydrogel beads formed are within 30-100 microns in size. The micrographs also indicate significant differences in the morphology of the hydrogel formed by the inverse emulsion polymerization technique compared to the bulk polymerization technique (in which no oil phase exits). The hydrogel prepared by the bulk technique has larger pores after lyophilization (10-20 microns in size). Such large pores lead to the burst release of a loaded molecules, such as an enzyme or other biologically active biomacromolecules.

### 17. Physicochemical Properties of BSA Laden Zwitterionic PSBMA Hydrogel

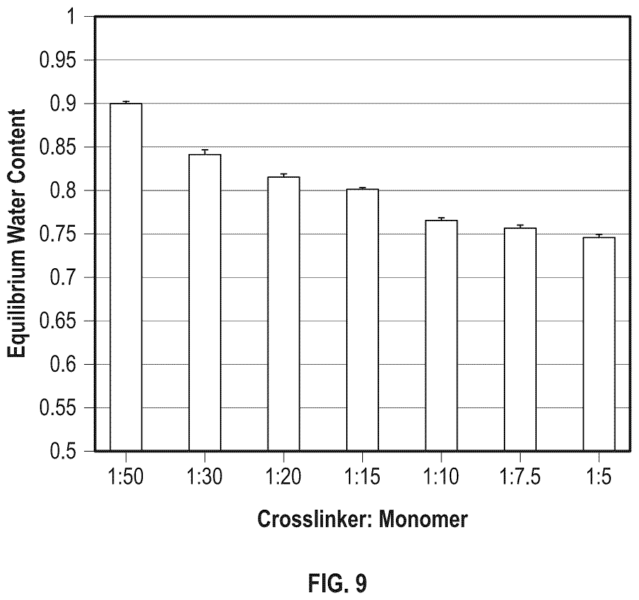

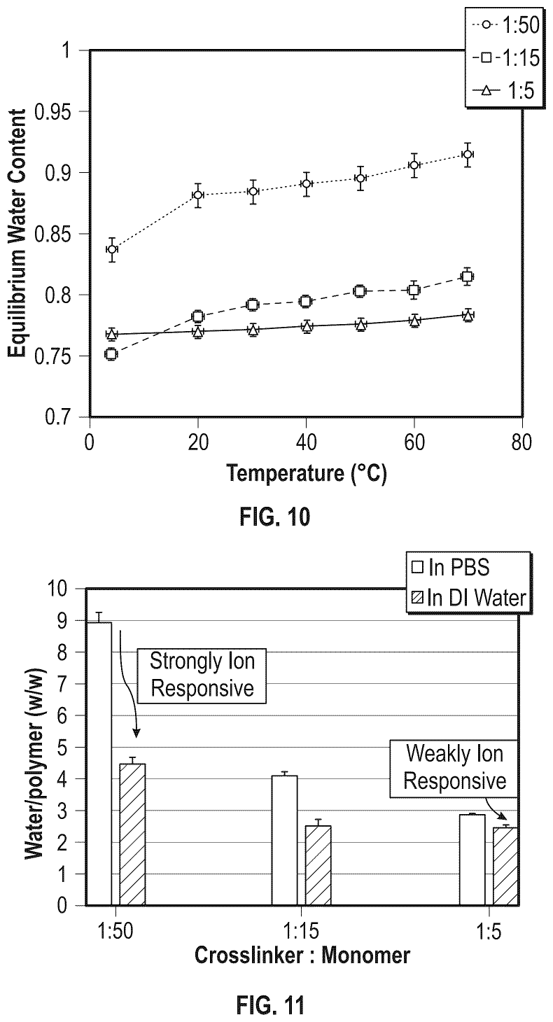

The water content of the different hydrogels was measured, and the results are presented in FIG. 9. The measured water contents of pSB hydrogels over a range of temperatures (FIG. 10) shows a positive temperature dependency behavior. The positive temperature dependency of water content for pSB is different from many previously studied hydrogels such as N,N-diethylacrylamide (PDEAM) or N-isopropylacrylamide (pNIPAAm) which have shown negative temperature dependency of swelling (lower critical solution temperature behavior). For PDEAM and pNIPAAm, at low temperatures, the gel is in its swollen state. For materials like PNIPAAm, the negative thermosensitivity is explained by hydrogen bond thermosensitivity (higher temperature, weaker the water-polymer interactions). As can be seen in the FIG. 11, the hydrogel lost its water content by changing the solvent from PBS to DI water.

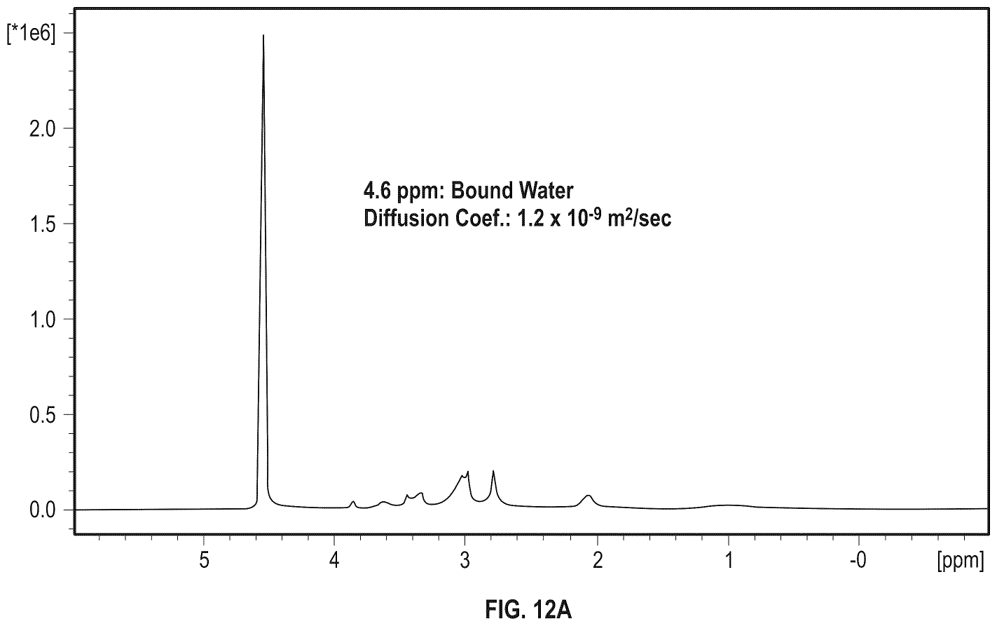

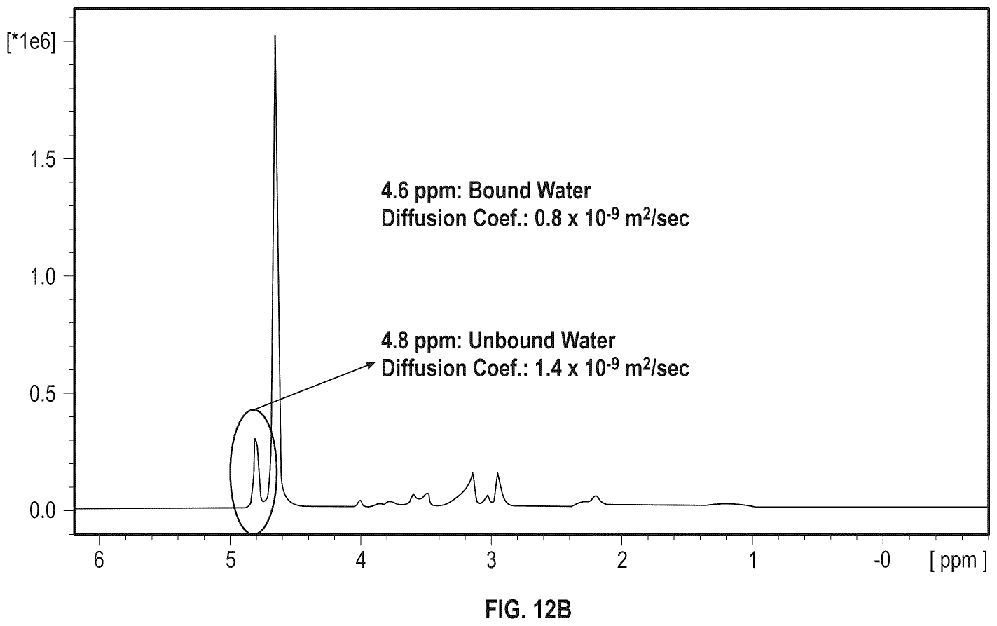

Diffusion NMR was performed to characterize directly the transport properties of the zwitterionic hydrogels. The measured, reduced diffusivity coefficients of different studied gels are presented in Table 1. As can been seen in the table, the effective self-diffusion of water (related to the mean square random displacement of water molecules) and the tortuosity of the zwitterionic hydrogel can be controlled by the crosslinking density. Water inside hydrogels exists in different microscopic states. The bound water is defined as the fraction of water that at any specific time is effectively immobilized by the hydrogel. The bound and unbound water within the hydrogel was also observed using ¹H NMR. FIGS. 12A-12B illustrates the ¹H NMR spectra of two different crosslinking densities of pSB hydrogel. The unbound/bound water ratio is also summarized in Table 1. The results indicate that at low crosslinking densities (1:50), all of the water molecules in the hydrogel are bound. In comparison, other hydrogels such as pHEMA (poly hydroxyethyl methacrylate) hydrogels can have up to half of associated water unbound. See, McConville, P.; Pope, J. M., A comparison of water binding and mobility in contact lens hydrogels from NMR measurements of the water self-diffusion coefficient. Polymer 2000, 41 (26), 9081-9088. The small amount of unbound water in pSB indicates the strong water-polymer association in the hydrogel. The high water content and high bound water ratio of pSB compared to other hydrogels can be attributed to the superhydrophilicity of pSB. For pSB hydrogels, the unbound/bound ratio can increase to 0.20 by increasing the extent of crosslinking. The direct relationship between unbound/bound water ratio and the crosslinking density indicates how the bis-acrylamide crosslinker disrupts the structure of the hydrogel.

TABLE 1
Measured diffusion coefficients of different studied hydrogels

| | H₂O/D₂O | | | Reduced | |
|---|---|---|---|---|---|
| | mixture | | | Diffusion Coef. | Reduced Diffusion |
| Gel crosslinked | diffusivity | | | (D_{eff}/D₀) | Coef. (D_{eff}/D₀) |
| density (molar | Coef. | | Unbound/bound | Zwitterionic | Poly Vinyl Alcohol |
| ratio) | (10⁹ m²/sec) | Tortuosity | water ratio | Gel | gels⁴⁸ |
| Low (1:50) | 1.424 | 1.30 | 0.000 | 0.77 | 0.79 |
| Medium-low | 1.200 | 1.54 | 0.004 | 0.65 | — |
| (1:30) | | | | | |
| Medium (1:15) | 1.010 | 1.81 | 0.103 | 0.55 | 0.65 |
| Medium-high | 0.888 | 2.08 | 0.139 | 0.48 | — |
| (1:10) | | | | | |
| High (1:5) | 0.796 | 2.33 | 0.170 | 0.43 | 0.61 |

### 15. Results—BSA Laden Zwitterionic PSBMA Hydrogel Post-Fabrication Protein Loading

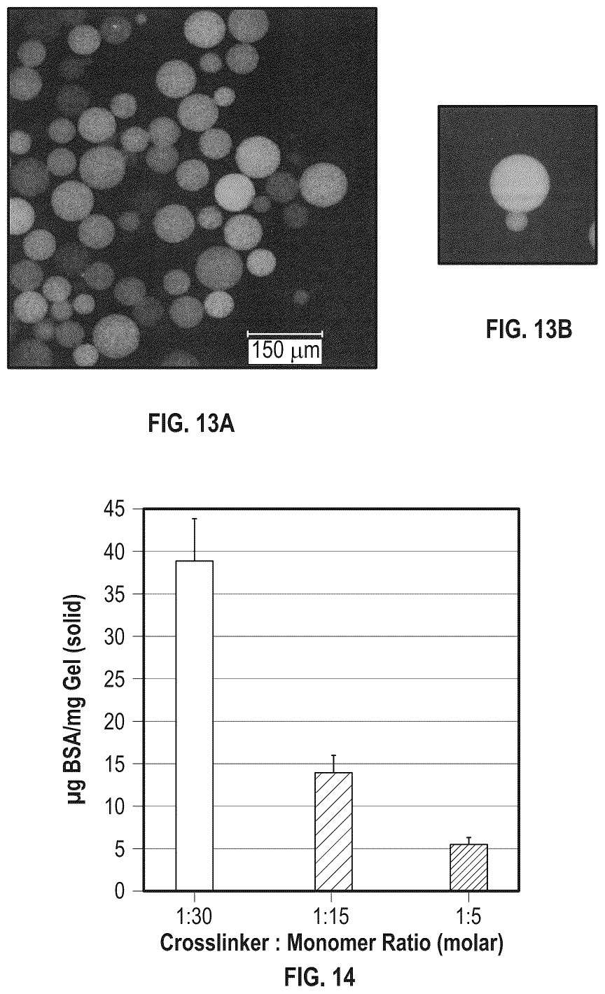

Unwanted chemical reactions between the hydrogel and the protein amino acid residues may be prevented by loading the protein after the hydrogel formation. FIGS. 13A-13B illustrates the confocal fluorescence microscopy of the hydrogels loaded with fluorescently tagged BSA. The amount of protein loaded per weight of dried hydrogel is illustrated in FIG. 14.

### 16. ChT Deactivation and Half-Life

The increased conformational stability and enhanced activity of i-ChT at elevated pH/temperatures suggested that the half-life of ChT should also be affected by the immobilization within the pCB hydrogel. As such, the permanent deactivation of the enzyme caused by denaturation/aggregation were studied at different temperatures and pHs and are presented in Tables 2 and 3, respectively. As shown in Table 2, while both free and immobilized ChT half-life decreased with increasing temperature, the i-ChT half-life is significantly improved at elevated temperatures. This lower denaturation/aggregation rate can be attributed to at least two factors. First, the smaller diffusivity inside the hydrogel (compared to free water) slows down molecular motion and direct protein-protein interactions. Second, with the ability of the zwitterions to increase the activation energy gaps in protein denaturing, the zwitterionic environment increases protein conformational stability. The increased stability of i-ChT within the zwitterionic hydrogel allows for the use of enzymes at elevated temperatures.

As can be seen in Table 3, the half-life of free and immobilized ChT decreased with increasing pH for the studied pH conditions. At neutral and basic pHs, the immobilized enzyme has significantly longer half-life. This is not true for acidic pHs; however, at a pH of 3, the half-life of the immobilized enzyme decreased by 24%. This is due to the effects of ChT lysine residues on ChT stability in acidic environments. A previous report indicated that at a pH of 3-4, repulsive electrostatic charges of lysine residues can prevent ChT aggregation (ChT has a net charge of +14 mV at pH 4). See, Rezaei-Ghaleh, N., et al., Thermal aggregation of α—chymotrypsin: role of hydrophobic and electrostatic interactions. Biophysical chemistry, 2008. 132(1): p. 23-32. As disclosed and claimed here, immobilization of the enzyme occurred due to a carboxylic acid-lysine residue reaction, and therefore some of the lysine residues were thereby neutralized. As such, a smaller half-life for the immobilized enzyme at pH 3 and 4 was observed.

TABLE 2
Half-life of chymotrypsin at pH of 8 and different temperatures.
The half-life measurements were carried out at conditions with
equal volumetric activity (0.15 mg/ml chymotrypsin in solution
and 30 mg/ml enzyme immobilized hydrogel suspension).

| | Half-life (min) | | % Change in |
|---|---|---|---|
| | | Immobilized | half-life after |
| Temperature (° C.) | Free Enzyme | Enzyme | immobilization |
| 30 | 4680 | 4692 | 0.3% |
| 40 | 512 | 2031 | 297% |
| 50 | 44 | 196 | 346% |
| 60 | 5 | 36 | 620% |

TABLE 3
Half-life of chymotrypsin in different pHs at T = 40°
C. The half-life measurements were carried out at conditions
with equal volumetric activity (0.15 mg/ml chymotrypsin in
solution and 30 mg/ml enzyme immobilized hydrogel suspension).

| | | | % Change in half- |
|---|---|---|---|
| | Half-life (min) | | life after |
| pH | Free Enzyme | Immobilized Enzyme | immobilization |
| 3 | 14850 | 11321 | −24% |
| 4 | 12956 | 10714 | −17% |
| 5 | 4667 | 8980 | 92% |
| 7 | 1500 | 3870 | 158% |
| 8 | 512 | 2031 | 297% |
| 9 | 115 | 822 | 615% |

The foregoing description provides illustration and description, but is not intended to be exhaustive or to limit the inventive concepts to the precise form disclosed. Modifications and variations are possible in light of the above teachings or may be acquired from practice of the methodologies set forth in the present disclosure.

Even though particular combinations of features and steps are recited in the claims and/or disclosed in the specification, these combinations are not intended to limit the disclosure. In fact, many of these features and steps may be combined in ways not specifically recited in the claims and/or disclosed in the specification. Although each dependent claim listed below may directly depend on only one other claim, the disclosure includes each dependent claim in combination with every other claim in the claim set.

1.-29. (canceled)

30. A composition comprising a synthesized zwitterionic microscale hydrogel bead loaded with at least one active biomacromolecule.

31. The composition of claim 30, wherein the active biomacromolecule is encapsulated within the synthesized zwitterionic microscale hydrogel bead.

32. The composition of claim 30, wherein the active biomacromolecule is immobilized by the synthesized zwitterionic microscale hydrogel bead.

33. The composition of claim 30, wherein the synthesized zwitterionic microscale hydrogel bead comprises a cross-linked polysulfobetaine methyl methacrylate (SBMA) polymer.

34. The composition of claim 30, wherein the synthesized zwitterionic microscale hydrogel bead comprises a covalently crosslinked zwitterionic polymer.

35. The composition of claim 34, wherein the covalently crosslinked zwitterionic polymer comprises a crosslink to monomer molar ratio in a range of from about 1:50 to about 1:5.

36. The composition of claim 30, wherein the synthesized zwitterionic microscale hydrogel bead comprises a crosslinked zwitterionic polymer, wherein at least some crosslinks within the crosslinked zwitterionic polymer are degradable.

37. The composition of claim 36, wherein the crosslinked zwitterionic polymer comprises a crosslink to monomer molar ratio in a range of from about 1:50 to about 1:5.

38. The composition of claim 30, wherein the synthesized zwitterionic microscale hydrogel bead has a size of no more than 200 microns.

39. The composition of claim 38, wherein the synthesized zwitterionic microscale hydrogel bead has a size of from about 1 to about 20 microns.

40. The composition of claim 30, wherein the at least one active biomacromolecule is selected from the group consisting of enzymes, cytokines, globular proteins, monoclonal antibodies, and combinations thereof.

41. The composition of claim 30, wherein the synthesized zwitterionic microscale hydrogel bead substantially retains a spherical shape after lyophilization and rehydration.

42. The composition of claim 41, wherein the synthesized zwitterionic microscale hydrogel bead substantially retains a free-flowing state when placed in a device for delivery to a target.

43. The composition of claim 30, wherein the at least one active biomacromolecule is chemically immobilized by the synthesized zwitterionic microscale hydrogel bead, wherein at least one of a melting point and a stability of the chemically immobilized at least one active biomacromolecule is increased over at least one of a melting point and a stability of a non-chemically immobilized form of the at least one active biomacromolecule, wherein the stability of the chemically immobilized or non-chemically immobilized forms of the at least one active biomacromolecule is selected from the group consisting of transient stability, physical stability, chemical stability, thermal stability, and combinations thereof.

44. The composition of claim 30, wherein the at least one active biomacromolecule is loaded in the synthesized zwitterionic microscale hydrogel bead at a per weight of the synthesized zwitterionic microscale hydrogel bead in a range of from about 5 micrograms/milligram to about 500 micrograms/milligram per weight of a dried amount of the synthesized zwitterionic microscale hydrogel bead having at least one active biomacromolecule loaded therein.

45. The composition of claim 30, wherein the composition comprises at least two synthesized zwitterionic microscale hydrogel beads loaded with at least one active biomacromolecule.

46. The composition of claim 45, wherein the composition is substantially monodispersed.

47. A composition comprising a synthesized zwitterionic microscale hydrogel bead, loaded with at least one active biomacromolecule selected from the group consisting of enzymes, cytokines, globular proteins, monoclonal antibodies, and combinations thereof.

48. The composition of claim 47, wherein the composition comprises at least two synthesized zwitterionic microscale hydrogel beads loaded with at least one active biomacromolecule.

49. The composition of claim 48, wherein the composition is substantially monodispersed.

50. The composition of claim 48, wherein the synthesized zwitterionic microscale hydrogel bead has a size of no more than 200 microns.

51. The composition of claim 50, wherein the synthesized zwitterionic microscale hydrogel bead has a size of from about 1 to about 20 microns.
